(19) United States
(12) Patent Application Publication
(10) Pub. No.: US 2025/0387361 A1
(43) Pub. Date: Dec. 25, 2025

(54) METHODS OF ADMINISTERING GAMMA-HYDROXYBUTYRATE COMPOSITIONS WITH DIVALPROEX SODIUM

(71) Applicant: Flamel Ireland Limited, Dublin (IE)

(72) Inventor: Julien Grassot, Lyon (FR)

(21) Appl. No.: 19/307,151

(22) Filed: Aug. 22, 2025

Related U.S. Application Data
(63) Continuation of application No. 17/502,562, filed on Oct. 15, 2021.
Application No. 17/502,562 is a continuation-in-part of application No. 17/231,455, filed on Apr. 15, 2021, now abandoned.
(60) Provisional application No. 63/010,974, filed on Apr. 16, 2020.

(51) Int. Cl. A61K 31/19; A61P 25/00

(52) U.S. Cl. CPC A61K 31/19; A61P 25/00

(57) ABSTRACT

Oral pharmaceutical compositions of gamma-hydroxybutyrate (GHB) suitable for concomitant administration with a dose of divalproex sodium (DVP) without materially altering the dosage amount of either drug are provided. Also provided are therapeutic uses of the compositions for the treatment of one or more symptoms of narcolepsy.

### CROSS-REFERENCE TO RELATED APPLICATIONS

This application is a continuation of U.S. application Ser. No. 17/502,562, filed Oct. 15, 2021 which is a continuation-in-part of U.S. application Ser. No. 17/231,455, filed Apr. 15, 2021, which claims priority to U.S. Provisional Application No. 63/010,974, filed Apr. 16, 2020.

### FIELD

The present invention relates to compositions for the treatment of narcolepsy, such as any of the symptoms of narcolepsy (e.g., cataplexy, excessive daytime sleepiness, disrupted nighttime sleep, hypnagogic hallucinations, or sleep paralysis) comprising gamma-hydroxybutyrate in a unit dose suitable for administration with divalproex sodium. The present invention also relates to modified release formulations of gamma-hydroxybutyrate having improved pharmacokinetic (PK) properties with concomitant administration of divalproex sodium.

### BACKGROUND

Narcolepsy is a devastating disabling condition. The cardinal symptoms are excessive daytime sleepiness (EDS), cataplexy (a sudden loss of muscle tone triggered by strong emotions, seen in approximately 60% of patients), hypnogogic hallucination (HH), sleep paralysis (SP), and disturbed nighttime/nocturnal sleep (DNS). Other than EDS, DNS is the most common symptom seen among narcolepsy patients.

One of the major treatments for narcolepsy is sodium oxybate, a neuroactive agent with a variety of Central Nervous System (CNS) pharmacological properties. The species is present endogenously in many tissues, where it acts as a neurotransmitter on a gamma-hydroxybutyrate (GHB) receptor (GHBR), and possesses neuromodulatory properties with significant effects on dopamine and gamma-Aminobutyric Acid (GABA). Studies have suggested that sodium oxybate improves Rapid Eye Movement Sleep (REM sleep, REMS) of narcoleptics in contrast to antidepressant drugs.

Sodium oxybate is also known as sodium 4-hydroxybutanoate, or gamma-hydroxybutyric acid sodium salt, and has the following chemical structure:

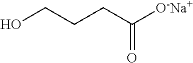

Sodium oxybate is marketed commercially in the United States as Xyrem®. The product is formulated as an immediate release liquid solution that is taken once immediately before bed, and a second time approximately 2.5 to 4 hours later, in equal doses. Sleep-onset may be dramatic and fast, and patients are advised to be sitting in bed when consuming the dose. The most commonly reported side effects are confusion, depressive syndrome, incontinence and sleepwalking.

One critical drawback of Xyrem® is the requirement to reduce the initial dosage of Xyrem if there is concomitant use with divalproex sodium (DVP). Specifically, Xyrem®'s label expressly advises “Concomitant use with Divalproex Sodium: an initial reduction in Xyrem® dose of at least 20% is recommended.” After a clinical trial for co-administration of Xyrem and divalproex sodium, the following language was added to the Xyrem label at section 2.4: “Pharmacokinetic and pharmacodynamic interactions have been observed when Xyrem is co administered with divalproex sodium. For patients already stabilized on Xyrem, it is recommended that addition of divalproex sodium should be accompanied by an initial reduction in the nightly dose of Xyrem by at least 20%. For patients already taking divalproex sodium, it is recommended that prescribers use a lower starting Xyrem dose when introducing Xyrem.” The medical problem cautioned against by the Xyrem® label and unaddressed by the prior art is pharmacokinetic and pharmacodynamic interactions when Xyrem® is co-administered with divalproex sodium. As noted in the Xyrem®s Drug Interactions section of the Prescribing Information, “Concomitant use of Xyrem with divalproex sodium resulted in a 25% mean increase in systemic exposure to Xyrem (AUC ratio range of 0.8 to 1.7) and in a greater impairment on some tests of attention and working memory.” As a practical matter, this requires prescribers to monitor patient response closely and adjust dose accordingly for concomitant use of Xyrem® and divalproex sodium. In addition, U.S. Pat. No. 8,772,306 to Jazz Pharmaceuticals teaches that the dosage amount of GHB must be decreased by at least 5% decrease when the patient is receiving a concomitant administration of valproate, an acid, salt, or mixture thereof (e.g. divalproex sodium).

Accordingly, there is a need for compositions of gamma-hydroxybutyrate that can be co-administered with divalproex sodium without having to reduce the dose of gamma-hydroxybutyrate and without compromising safety or efficacy.

### SUMMARY OF THE INVENTION

In an aspect, the present disclosure encompasses a method of treating narcolepsy (e.g., one or more symptoms of narcolepsy) by administering a GHB composition concomitantly with divalproex sodium (DVP) without reducing the dose of GHB. For example, a method for treating a patient suffering from excessive daytime sleepiness (EDS), disrupted nighttime sleep (DNS), cataplexy, hypnagogic hallucinations, or sleep paralysis may include orally administering to the patient a full dosage amount of a pharmaceutical composition comprising GHB and concomitantly administering a full dosage amount of a pharmaceutical composition comprising DVP. In some examples, the dosage of the GHB composition is not reduced in response to the concomitant administration of DVP and/or the dosage of the DVP is not reduced in response to the concomitant administration of GHB composition. In other examples, where the dosage of one or both GHB and DVP is reduced, such reduction is by less than 5% of the full dosage amount in response to the concomitant administration of DVP.

Further provided herein is an oral pharmaceutical composition of GHB for the treatment of narcolepsy (e.g., one or more symptoms of narcolepsy) that may be concomitantly administered with DVP. In some examples, the dosage of the GHB composition is not reduced in response to the concomitant administration of DVP, and the dosage of the DVP is not reduced in response to the concomitant administration of GHB composition. In other words, both the dosage amounts of the GHB composition and the DVP are not reduced at all when coadministered. In other examples, the dosage of one or both the GHB composition and the DVP is reduced by less than 5% of the full dosage amount when coadministered.

Other aspects and iterations of the invention are described more thoroughly below.

### DESCRIPTION OF THE FIGURES

The accompanying drawings, which are incorporated in and constitute a part of this specification, illustrate several embodiments of the invention and together with the description serve to explain the principles of the invention.

FIG. 1A is a mean concentration versus time curve for 6 g FT218 administered alone and with DVP in the evening.

FIG. 1B is a series of individual profiles in a mean concentration versus time curve for 6 g FT218 administered alone and with DVP in the evening.

FIG. 2A shows a comparison of mean Tₘₐₓ for 6 g FT218 administered alone and with DVP in the evening.

FIG. 2B shows a comparison of mean Cₘₐₓ for 6 g FT218 administered alone and with DVP in the evening.

FIG. 2C shows a comparison of mean AUC_{inf} for 6 g FT218 administered alone and with DVP in the evening.

FIG. 3A is a mean concentration versus time curve for DVP administered alone and with FT218 in the evening.

FIG. 3B is a series of individual profiles in a mean concentration versus time curve for DVP administered alone and with FT218 in the evening.

FIG. 4A is a mean concentration versus time curve for 6 g FT218 administered alone and with DVP in the morning.

FIG. 4B is a series of individual profiles in a mean concentration versus time curve for 6 g FT218 administered alone and with DVP in the morning.

FIG. 5A shows a comparison of mean Tₘₐₓ for 6 g FT218 administered alone and with DVP in the morning.

FIG. 5B shows a comparison of mean Cₘₐₓ for 6 g FT218 administered alone and with DVP in the morning.

FIG. 5C shows a comparison of mean AUC_{inf} for 6 g FT218 administered alone and with DVP in the morning.

FIG. 6A is a mean concentration versus time curve for DVP administered alone and with FT218 in the morning.

FIG. 6B is a series of individual profiles in a mean concentration versus time curve for DVP administered alone and with FT218 in the morning.

FIG. 7 is a mean concentration versus time curve for 6 g FT218 administered alone and with DVP either in the morning (DDI #1) or in the evening (DDI #2).

### DETAILED DESCRIPTION

The present invention may be understood more readily by reference to the following detailed description of embodiments of the formulation, methods of treatment using some embodiments of the formulation, and the Examples included therein.

### Definitions and Use of Terms

Wherever an analysis or test is required to understand a given property or characteristic recited herein, it will be understood that the analysis or test is performed in accordance with applicable guidances, draft guidances, regulations and monographs of the United States Food and Drug Administration (“FDA”) and United States Pharmacopoeia (“USP”) applicable to drug products in the United States in force as of Nov. 1, 2015 unless otherwise specified. Clinical endpoints may be judged with reference to standards adopted by the American Academy of Sleep Medicine, including standards published at C Iber, S Ancoli-Israel, A Chesson, S F Quan. The AASM Manual for the Scoring of Sleep and Associated Events. Westchester, IL: American Academy of Sleep Medicine; 2007.

When a pharmacokinetic comparison is made between a formulation described or claimed herein and a reference product, it will be understood that the comparison is performed in a suitable designed cross-over trial, although it will also be understood that a cross-over trial is not required unless specifically stated. It will also be understood that the comparison may be made either directly or indirectly. For example, even if a formulation has not been tested directly against a reference formulation, it can still satisfy a comparison to the reference formulation if it has been tested against a different formulation, and the comparison with the reference formulation may be deduced therefrom.

As used in this specification and in the claims which follow, the singular forms “a,” “an” and “the” include plural referents unless the context dictates otherwise. Thus, for example, reference to “an ingredient” includes mixtures of ingredients, reference to “an active pharmaceutical agent” includes more than one active pharmaceutical agent, and the like.

“Bioavailability” means the rate and extent to which the active ingredient or active moiety is absorbed from a drug product and becomes available at the site of action.

“Relative bioavailability” or “Rel BA” or “RBA” means the percentage of mean AUC_{inf} of the tested product relative to the mean AUC_{inf} of the reference product for an equal total dose. Unless otherwise specified, relative bioavailability refers to the percentage of the mean AUC_{inf} observed for a full dose of the test product co-administered with divalproex sodium relative to the mean AUC_{inf} observed for an equal total dose of the test product without administration of divalproex sodium.

“Bioequivalence” means the absence of a significant difference in the rate and extent to which the active ingredient or active moiety in pharmaceutical equivalents or pharmaceutical alternatives become available at the site of drug action when administered at the same molar dose under similar conditions in an appropriately designed study. In some examples, “bioequivalence range” means a test composition/condition has a PK value within 80%-125% of the PK value for a reference composition/condition.

When ranges are given by specifying the lower end of a range separately from the upper end of the range, it will be understood that the range may be defined by selectively combining any one of the lower end variables with any one of the upper end variables that is mathematically and physically possible. Thus, for example, if a formulation may contain from 1 to 10 weight parts of a particular ingredient, or 2 to 8 parts of a particular ingredient, it will be understood that the formulation may also contain from 2 to 10 parts of the ingredient. In like manner, if a formulation may contain greater than 1 or 2 weight parts of an ingredient and up to 10 or 9 weight parts of the ingredient, it will be understood that the formulation may contain 1-10 weight parts of the ingredient, 2-9 weight parts of the ingredient, etc. unless otherwise specified, the boundaries of the range (lower and upper ends of the range) are included in the claimed range.

When used herein the term “about” or “substantially” or “approximately” will compensate for variability allowed for in the pharmaceutical industry and inherent in pharmaceutical products, such as differences in product strength due to manufacturing variation and time-induced product degradation. The term allows for any variation which in the practice of pharmaceuticals would allow the product being evaluated to be considered bioequivalent to the recited strength, as described in FDA's March 2003 Guidance for Industry on BIOAVAILABILITY AND BIOEQUIVALENCE STUDIES FOR ORALLY ADMINISTERED DRUG PRODUCTS—GENERAL CONSIDERATIONS.

When used herein the term “gamma-hydroxybutyrate” or GHB, unless otherwise specified, refers to the free base of gamma-hydroxybutyrate and any pharmaceutical composition that releases free GHB base into the bloodstream of a patient, including a pharmaceutically acceptable salt of gamma-hydroxybutyric acid, a prodrug of gamma-hydroxybutyrate, their hydrates, solvates, complexes, or tautomer forms, and combinations or mixtures thereof. Gamma-hydroxybutyric acid salts may be selected from the sodium salt of gamma-hydroxybutyric acid or sodium oxybate, the potassium salt of gamma-hydroxybutyric acid, the magnesium salt of gamma-hydroxybutyric acid, the calcium salt of gamma-hydroxybutyric acid, the lithium salt of gamma-hydroxybutyric, the tetra ammonium salt of gamma-hydroxybutyric acid or any other pharmaceutically acceptable salt forms of gamma-hydroxybutyric acid.

When used herein the term “divalproex sodium” or DVP, unless otherwise specified may include divalproex sodium, divalproic acid, valproic acid, valproate, an acid or salt of valproate, or a monocarboxylate transporter.

As used herein, the term “full dose” or “full dosage” refers to the dosage amount that would be administered to the patient without co-administration. For example, a full dosage of the GHB composition refers to the dosage that would be administered to the patient without co-administration of DVP and a full dosage of DVP refers to the dosage that would be administered to the patient without co-administration with the GHB composition.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use. The term “formulation” or “composition” refers to the quantitative and qualitative characteristics of a drug product or dosage form prepared in accordance with the current invention.

As used herein the doses and strengths of gamma-hydroxybutyrate are expressed in equivalent-gram (g) weights of sodium oxybate unless stated expressly to the contrary. Thus, when considering a dose of gamma-hydroxybutyrate other than the sodium salt of gamma-hydroxybutyrate, one must convert the recited dose or strength from sodium oxybate to the gamma-hydroxybutyrate under evaluation. Thus, if an embodiment is said to provide a 4.5 g dose of gamma-hydroxybutyrate, because the form of gamma-hydroxybutyrate is not specified, it will be understood that the dose encompasses a 4.5 g dose of sodium oxybate, a 5.1 g dose of potassium gamma-hydroxybutyrate (assuming a 126.09 g/mol MW for sodium oxybate and a 142.20 g/mol MW for potassium gamma-hydroxybutyrate), and a 3.7 g dose of the free base (assuming a 126.09 g/mol MW for sodium oxybate and a 104.1 g/mol MW for the free base of gamma-hydroxybutyrate), or by the weight of any mixture of salts of gamma-hydroxybutyric acid that provides the same amount of GHB as 4.5 g of sodium oxybate.

As used herein “microparticle” means any discreet particle of solid material. The particle may be made of a single material or have a complex structure with core and shells and be made of several materials. The terms “microparticle”, “particle”, “microspheres” or “pellet” are interchangeable and have the same meaning. Unless otherwise specified, the microparticle has no particular particle size or diameter and is not limited to particles with volume mean diameter D(4,3) below 1 mm.

As used herein, the “volume mean diameter D(4,3)” is calculated according to the following formula:

$$D ( 4 , 3 ) = \sum ( d 4 i \cdot ni ) / \sum ( d 3 i \cdot ni )$$

wherein the diameter d of a given particle is the diameter of a hard sphere having the same volume as the volume of that particle.

As used herein, the terms “composition”, “oral composition”, “oral pharmaceutical composition”, “finished composition”, “finished formulation” or “formulation” are interchangeable and designate the composition of gamma-hydroxybutyrate comprising modified release microparticles of gamma-hydroxybutyrate, immediate release microparticles of gamma-hydroxybutyrate, and any other excipients. The composition may be described as extended release, delayed release, or modified release.

As used herein, “immediate release” means release of the major part of gamma-hydroxybutyrate over a relatively short period, e.g. at least 75% of the AP is released in 0.75 h, for example, in 30 min.

As used herein, an “immediate release (IR) portion” of a formulation includes physically discreet portions of a formulation, mechanistically discreet portions of a formulation, and pharmacokinetically discreet portions of a formulation that lend to or support a defined IR pharmacokinetic characteristic. Thus, for example, any formulation that releases active ingredient at the rate and extent required of the immediate release portion of the formulations of the present invention includes an “immediate release portion,” even if the immediate release portion is physically integrated in what might otherwise be considered an extended release formulation. Thus, the IR portion may be structurally discreet or structurally indiscreet from (i.e. integrated with) the MR portion. In an embodiment, the IR portion and MR portion are provided as particles, and in other embodiments the IR portion and MR portion are provided as particles discreet from each other.

As used here in, “immediate release formulation” or “immediate release portion” refers to a composition that releases at least 80% of its gamma-hydroxybutyrate in 1 hour when tested in a dissolution apparatus 2 according to USP 38<711> in a 0.1N HCl dissolution medium at a temperature of 37° C. and a paddle speed of 75 rpm.

In like manner, a “modified-release (MR) portion” includes that portion of a formulation or dosage form that lends to or supports a particular MR pharmacokinetic characteristic, regardless of the physical formulation in which the MR portion is integrated. The modified release drug delivery systems are designed to deliver drugs at a specific time or over a period of time after administration, or at a specific location in the body. The USP defines a modified release system as one in which the time course or location of drug release or both, are chosen to accomplish objectives of therapeutic effectiveness or convenience not fulfilled by conventional IR dosage forms. More specifically, MR solid oral dosage forms include extended release (ER) and delayed-release (DR) products. A DR product is one that releases a drug all at once at a time other than promptly after administration. Typically, coatings (e.g., enteric coatings) are used to delay the release of the drug substance until the dosage form has passed through the acidic medium of the stomach. An ER product is formulated to make the drug available over an extended period after ingestion, thus allowing a reduction in dosing frequency compared to a drug presented as a conventional dosage form, e.g. a solution or an immediate release dosage form. For oral applications, the term “extended-release” is usually interchangeable with “sustained-release”, “prolonged-release” or “controlled-release”.

Traditionally, extended-release systems provided constant drug release to maintain a steady concentration of drug. For some drugs, however, zero-order delivery may not be optimal and more complex and sophisticated systems have been developed to provide multi-phase delivery. One may distinguish among four categories of oral MR delivery systems: (1) delayed-release using enteric coatings, (2) site-specific or timed release (e.g. for colonic delivery), (3) extended-release (e.g., zero-order, first-order, biphasic release, etc.), and (4), programmed release (e.g., pulsatile, delayed extended release, etc.) See Modified Oral Drug Delivery Systems at page 34 in Gibaldi's DRUG DELIVERY SYSTEMS IN PHARMACEUTICAL CARE, AMERICAN SOCIETY OF HEALTH-SYSTEM PHARMACISTS, 2007 and Rational Design of Oral Modified-release Drug Delivery Systems at page 469 in DEVELOPING SOLID ORAL DOSAGE FORMS: PHARMACEUTICAL THEORY AND PRACTICE, Academic Press, Elsevier, 2009. As used herein, “modified release formulation” or “modified release portion” in one embodiment refers to a composition that releases its gamma-hydroxybutyrate according a multiphase delivery that is comprised in the fourth class of MR products, e.g. delayed extended release. As such it differs from the delayed release products that are classified in the first class of MR products.

As used herein the terms “coating”, “coating layer,” “coating film,” “film coating” and like terms are interchangeable and have the same meaning. The terms refer to the coating applied to a particle comprising the gamma-hydroxybutyrate that controls the modified release of the gamma-hydroxybutyrate.

A “similar PK profile”, a “substantially similar PK profile”, or “comparable bioavailability” means that the mean AUC_{inf} of a test product co-administered with divalproex sodium is from 80% to 125% of the mean AUC_{inf} of same dosage of the test product administered alone in a suitably designed cross-over trial, the mean plasma concentration at 8 hours (C₈ₕ) of the test product co-administered with divalproex sodium is from 40% to 130% of the mean Csh of the reference product administered alone, and/or that the maximum plasma concentration (Cₘₐₓ) of the test product co-administered with divalproex sodium is from 50% to 140% of the Cₘₐₓ of the reference product administered alone.

As used herein, “dose proportional” occurs when increases in the administered dose are accompanied by proportional increases in the PK profile, such as the AUC or Cₘₐₓ.

A “concomitant PK profile” means the mean AUC_{inf}, the mean plasma concentration at 8 hours (C₈ₕ), and/or the maximum plasma concentration (Cₘₐₓ) of the composition when co-administered with divalproex sodium.

A “standard PK profile” means the mean AUC_{inf}, the mean plasma concentration at 8 hours (C₈ₕ), and/or the maximum plasma concentration (Cₘₐₓ) of the composition when administered alone (i.e. without co-administration with divalproex sodium).

One or more symptoms of narcolepsy include excessive daytime sleepiness (EDS), disrupted nighttime sleep (DNS), cataplexy, hypnagogic hallucinations, and sleep paralysis. Type 1 Narcolepsy (NT1) refers to narcolepsy characterized by excessive daytime sleepiness (“EDS”) and cataplexy. Type 2 Narcolepsy (NT2) refers to narcolepsy characterized by excessive daytime sleepiness without cataplexy. A diagnosis of narcolepsy (with or without cataplexy) may be confirmed by one or a combination of (i) an overnight polysomnogram (PSG) and a Multiple Sleep Latency Test (MSLT) performed within the last 2 years, (ii) a full documentary evidence confirming diagnosis from the PSG and MSLT from a sleep laboratory must be made available, (iii) current symptoms of narcolepsy including: current complaint of EDS for the last 3 months (ESS greater than 10), (iv) mean MWT less than 8 minutes, (v) mean number of cataplexy events of 8 per week on baseline Sleep/Cataplexy Diary, and/or (vi) presence of cataplexy for the last 3 months and 28 events per week during screening period.

Unless otherwise specified herein, percentages, ratios and numeric values recited herein are based on weight; averages and means are arithmetic means; all pharmacokinetic measurements based on the measurement of bodily fluids are based on plasma concentrations.

It will be understood, when defining a composition by its pharmacokinetic or dissolution properties herein, that the formulation can in the alternative be defined as “means for” achieving the recited pharmacokinetic or dissolution properties. Thus, a formulation in which the modified release portion releases less than 20% of its gamma-hydroxybutyrate at one hour can instead be defined as a formulation comprising “means for” or “modified release means for” releasing less than 20% of its gamma-hydroxybutyrate at one hour. It will be further understood that the structures for achieving the recited pharmacokinetic or dissolution properties are the structures described in the examples hereof that accomplish the recited pharmacokinetic or dissolution properties.

### Oral Pharmaceutical Composition for Concomitant Administration with Divalproex Sodium

As the prior art demonstrates, it is extremely difficult to find a sodium oxybate formulation that may be concomitantly administered with divalproex sodium without reducing the dosage of sodium oxybate. It is also difficult to find a sodium oxybate formulation that when concomitantly administered with divalproex sodium has pharmacokinetic properties comparable to the sodium oxybate formulation without concomitant administration of divalproex sodium. The prior art, including the label for Xyrem, clearly teaches away from co-administering sodium oxybate and divalproex sodium at full doses. In fact, the label for Xyrem includes multiple statements recommending a reduction in the dose of Xyrem by at least 20% when co-administered with divalproex sodium based on a clinical trial finding “concomitant use of Xyrem with divalproex sodium resulted in a 25% mean increase in systemic exposure to Xyrem”.

The inventors have discovered a novel relationship between in vivo gamma-hydroxybutyrate absorption of modified release particles and the effect of divalproex sodium on the absorption of gamma-hydroxybutyrate which permits, for the first time, a full dose of a composition of gamma-hydroxybutyrate that may be concomitantly administered with divalproex sodium that approximates the bioavailability of the same composition of gamma-hydroxybutyrate at the same dose without administration of divalproex sodium, and that does so across a range of therapeutic doses. The dose of divalproex sodium administered may be a full dose that would be administered without administration of gamma-hydroxybutyrate.

Provided herein is an oral pharmaceutical composition for the treatment of narcolepsy, such as one or more symptoms of narcolepsy (e.g., excessive daytime sleepiness (EDS), disrupted nighttime sleep (DNS), cataplexy, hypnagogic hallucinations, and/or sleep paralysis) that includes gamma-hydroxybutyrate in a unit dose suitable for concomitant administration with divalproex sodium. In various embodiments, the composition may include gamma-hydroxybutyrate in an extended-release formulation, delayed release formulation, or modified release formulation.

The Xyrem® label indicates that there is a drug-drug interaction between Xyrem® and divalproex sodium, such that the divalproex sodium impacts the bioavailability of the Xyrem®, resulting in a recommendation that the Xyrem® dosage should be reduced when co-administered with divalproex sodium. In addition, the Xyrem risk evaluation and mitigation strategy (REMS) Program is a monitoring component that requires specific risk mitigation actions for the DDI between Xyrem and divalproex sodium. The FDA has concluded that information regarding the DDI with divalproex sodium cannot be “carved out” from an ANDA for a sodium oxybate product referencing Xyrem®. Based on literature data on GHB and competitive elimination pathway with divalproate, similar results as Xyrem® would have been expected. However, surprisingly, the gamma-hydroxybutyrate composition may be co-administered with divalproex sodium without being significantly impacted by the divalproex sodium. The gamma-hydroxybutyrate composition is a once daily composition with two waves of release of GHB. Without being limited to any particular theory, the two wave release of the gamma-hydroxybutyrate composition may allow for co-administration with divalproex sodium without reducing the GHB dosage. For example, the first wave may behave similarly as the reference Xyrem, while the second wave, releasing latter in the gastrointestinal tract may skip a part of the competition on the metabolic pathway, resulting in a lower interaction effect with divalproex sodium.

In an embodiment, the gamma-hydroxybutyrate composition may be co-administered with divalproex sodium without having to reduce the dosage of the gamma-hydroxybutyrate composition at any time during administration. In an embodiment, the divalproex sodium may be co-administered with the gamma-hydroxybutyrate composition without having to reduce the dosage of the divalproex sodium at any time during administration. For example, the gamma-hydroxybutyrate composition may be administered to a patient in need thereof that is already taking divalproex sodium without reducing the dosage of the gamma-hydroxybutyrate composition compared to the dosage that would be administered if the patient were not taking divalproex sodium. In another example, divalproex sodium may be administered to a patient in need thereof that is already taking the gamma-hydroxybutyrate composition without reducing the dosage of the gamma-hydroxybutyrate composition the patent is currently taking. Because the present gamma-hydroxybutyrate composition may be co-administered with divalproex sodium without reducing the dosage of either composition, there may be a reduced need for a monitoring component or no monitoring component. For example, the gamma-hydroxybutyrate composition may not need a prescriber information/brochure and/or patient counseling information relating to co-administration with divalproex sodium.

The Xyrem® label explicitly teaches that Xyrem® should not be co-administered with divalproex sodium without reducing the dosage of Xyrem® by 20%, as the divalproex sodium increases the systemic exposure of gamma-hydroxybutyrate from Xyrem beyond 25% of systemic exposure when Xyrem® is administered alone. Contrary to this, concomitant use of the present gamma-hydroxybutyrate composition with divalproex sodium may result in a lower change in systemic exposure to the gamma-hydroxybutyrate composition, as compared to concomitant administration of Xyrem® and divalproex sodium. For example, concomitant use of the gamma-hydroxybutyrate composition with divalproex sodium may result in a less than 25% mean increase in systemic exposure to the gamma-hydroxybutyrate composition. In some examples, concomitant use of the gamma-hydroxybutyrate composition with divalproex sodium may result in a less than 15% mean increase in systemic exposure to the gamma-hydroxybutyrate composition. In other examples, concomitant use of the gamma-hydroxybutyrate composition with divalproex sodium may result in a less than 5% mean increase in systemic exposure to the gamma-hydroxybutyrate composition. In at least one example, concomitant use of the gamma-hydroxybutyrate composition with divalproex sodium may result in no change in systemic exposure to the gamma-hydroxybutyrate composition.

The Xyrem® label also explicitly teaches that Xyrem® co-administered with divalproex sodium can result in impairment on some tests of attention and working memory. Surprisingly, concomitant use of the gamma-hydroxybutyrate composition with divalproex sodium may result in fewer side effects, as compared to concomitant administration of Xyrem® and divalproex sodium. For example, concomitant use of the gamma-hydroxybutyrate composition with divalproex sodium may result in less impairment on some tests of attention and working memory, as compared to concomitant administration of Xyrem® and divalproex sodium. In other examples, patients may not reduce the dosage without risking side effects of GHB overdosage.

The oral pharmaceutical composition of gamma-hydroxybutyrate may be in a unit dose suitable for co-administration with divalproex sodium without reducing the dosage of gamma-hydroxybutyrate for the treatment of narcolepsy or one or more symptoms of narcolepsy (e.g., one or more symptoms of narcolepsy selected from excessive daytime sleepiness (EDS), disrupted nighttime sleep (DNS), cataplexy, hypnagogic hallucinations, and sleep paralysis) in a human subject in need thereof. In some embodiments, the oral pharmaceutical composition may be effective to treat narcolepsy, cataplexy, or excessive daytime sleepiness in a human subject in need thereof. In some examples, the human subject may be a human patient. In any of the embodiments provided herein, the formulation may be effective to treat narcolepsy Type 1 or Type 2. The treatment of narcolepsy may be defined as reducing excessive daytime sleepiness, reducing the frequency of cataplectic attacks, reducing disrupted nighttime sleep, reducing hypnagogic hallucinations, or reducing sleep paralysis. In various embodiments, the composition is sufficient to be administered once daily. For example, the composition may be sufficient to administer in the morning or at night concomitant with divalproex sodium. The formulation is also effective to induce sleep for at least 6 to 8 consecutive hours. In one embodiment, the composition co-administered with divalproex sodium is effective to induce sleep for at least 8 consecutive hours. In various embodiments, the formulation is effective to induce sleep for at least 6 hours, at least 7 hours, at least 8 hours, at least 9 hours, or at least 10 hours. In other embodiments, the formulation is effective to induce sleep for up to 6 hours, up to 7 hours, up to 8 hours, up to 9 hours, or up to 10 hours.

The compositions of gamma-hydroxybutyrate may have both immediate release and modified release portions. The release of gamma-hydroxybutyrate from the immediate release portion is practically uninhibited, and occurs almost immediately in 0.1N hydrochloric acid dissolution medium. In contrast, while the modified release portion also may release its gamma-hydroxybutyrate almost immediately when fully triggered, the release is not triggered until a predetermined lag-time or the drug is subjected to a suitable dissolution medium such as a phosphate buffer pH 6.8 dissolution medium. Without wishing to be bound by any theory, it is believed that divalproex sodium may have no or low impact on the modified release portion of the composition, as the gamma-hydroxybutyrate from the modified release portion is absorbed in the latter part of the gastro-intestinal tract.

In any of these embodiments, the composition may include immediate release and modified release portions, where the modified release portion includes gamma hydroxybutyrate particles coated by a polymer carrying free carboxylic groups and a hydrophobic compound having a melting point equal or greater than 40° C., and the ratio of gamma-hydroxybutyrate in the immediate release portion and the modified release portion is from 10/90 to 65/35. The polymers comprising free carboxylic groups may have a pH dissolution trigger of from 5.5 to 6.97 and may be methacrylic acid copolymers having a pH dissolution trigger of from 5.5 to 6.97.

In various embodiments, the composition includes gamma-hydroxybutyrate present in a unit dose of at least 4.5 g, at least 6.0 g, at least 7.5 g, or at least 9.0 g. In some embodiments, the oral pharmaceutical composition of gamma-hydroxybutyrate may be administered as a once-daily dose concomitantly with a dose of divalproex sodium. In an example, the once-daily dose of the gamma-hydroxybutyrate is administered as a 6 g dose. The once-daily dose of the gamma-hydroxybutyrate may be administered once nightly. In an example, the once-nightly dose of the gamma-hydroxybutyrate is administered as a 6 g dose. The dose range of divalproex sodium ER is 10 to 60 mg/kg body weight per day. In some examples, the divalproex sodium is administered up to a daily dose of 60 mg/kg/day. In other examples, the divalproex sodium is administered at a dose of 1250 mg/day.

### Pharmacokinetics

The composition may provide a substantially similar concomitant PK profile and standard PK profile when the gamma-hydroxybutyrate composition is administered at the same dose. In some embodiments, the concomitant administration of gamma-hydroxybutyrate and divalproex sodium provides a substantially bioequivalent PK profile as compared to administration of an equal dose of the gamma-hydroxybutyrate composition in the absence of the concomitant administration of divalproex sodium.

In an embodiment, compositions of gamma-hydroxybutyrate co-administered with divalproex sodium may roughly approximate the bioavailability of an equal dose of the gamma-hydroxybutyrate composition without divalproex sodium, across the entire therapeutic range of gamma-hydroxybutyrate doses.

In an embodiment, there is no significant reduction in safety or efficacy to a patient following co-administration of the composition with divalproex sodium. For example, the safety profile for co-administration of the gamma-hydroxybutyrate composition and divalproex sodium may be consistent with what is known for sodium oxybate.

In another embodiment, the compositions of gamma-hydroxybutyrate may allow co-administration with divalproex sodium without a reduction in the dosage of gamma-hydroxybutyrate as compared to the commercial treatment Xyrem® which requires a reduction in the Xyrem® dosage by at least 5% when co-administered with divalproex sodium. In some examples, the dosage of the gamma-hydroxybutyrate composition is reduced by less than 5% in response to the concomitant administration of DVP.

In another embodiment, divalproex sodium may be co-administered with the compositions of gamma-hydroxybutyrate without a reduction in the dosage of divalproex sodium.

In other embodiments, the compositions of gamma-hydroxybutyrate may be co-administered with divalproex with improved dissolution and pharmacokinetic profiles compared to co-administration of Xyrem® and divalproex without reducing the Xyrem® dosage.

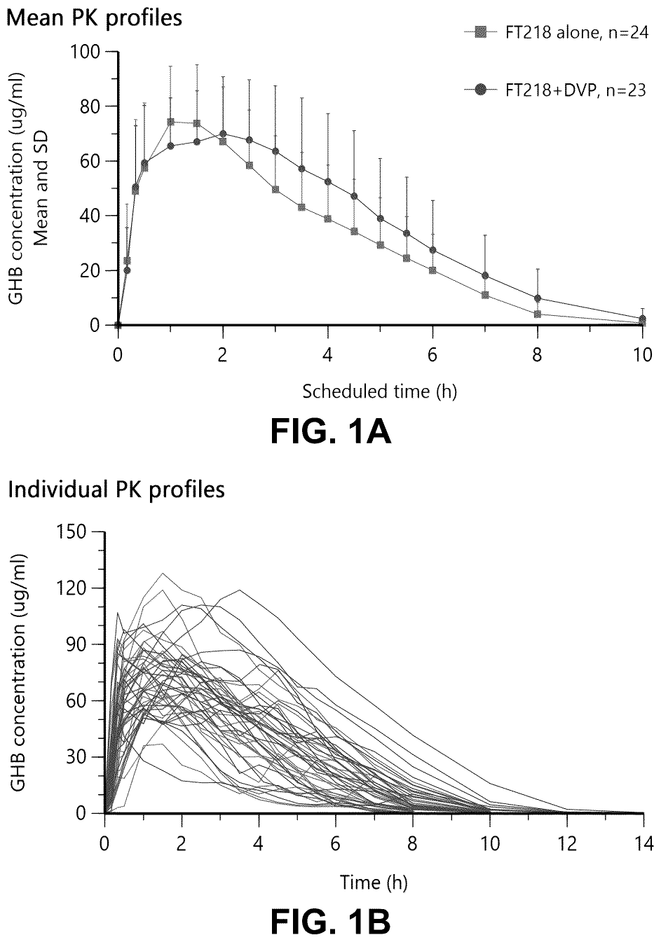

The compositions of gamma-hydroxybutyrate may also be defined by the concentration/time curves that they produce when tested according to the Examples. An embodiment of the composition of gamma-hydroxybutyrate yields a plasma concentration versus time curve when administered at a strength of 6 g concomitantly with divalproex sodium substantially as depicted in FIGS. 1A and 1B.

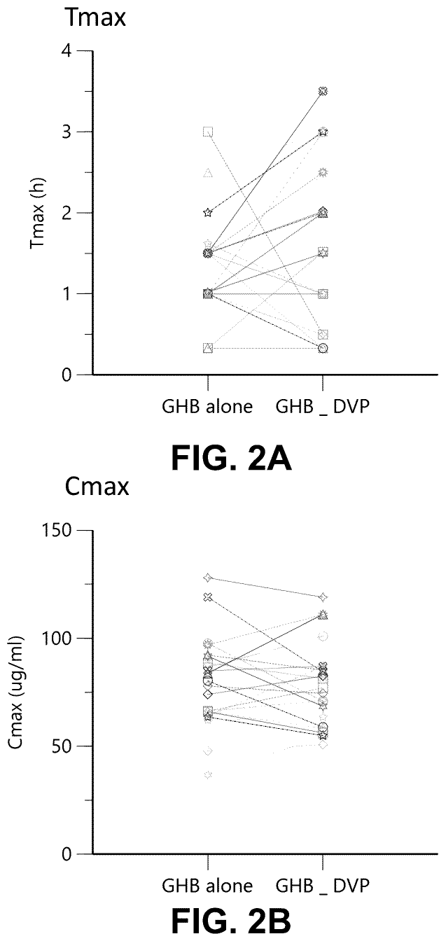

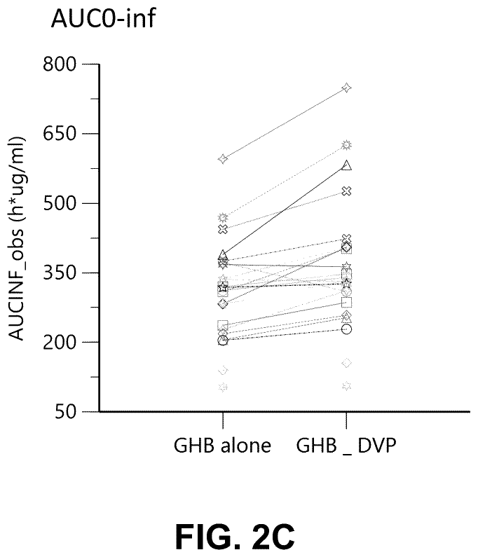

In an embodiment, concomitant administration of the gamma-hydroxybutyrate composition and divalproex sodium provides a Tₘₐₓ bioequivalent to a Tₘₐₓ of the same dosage of the gamma-hydroxybutyrate composition alone, as depicted in FIG. 2A. In another embodiment, concomitant administration of the gamma-hydroxybutyrate composition and divalproex sodium provides a Cₘₐₓ bioequivalent to a Cₘₐₓ of the same dosage of the gamma-hydroxybutyrate composition alone, as depicted in FIG. 2B. In an embodiment, concomitant administration of the gamma-hydroxybutyrate composition and divalproex sodium provides an AUC_{inf} bioequivalent to an AUC_{inf} of the same dosage of the gamma-hydroxybutyrate composition alone, as depicted in FIG. 2C.

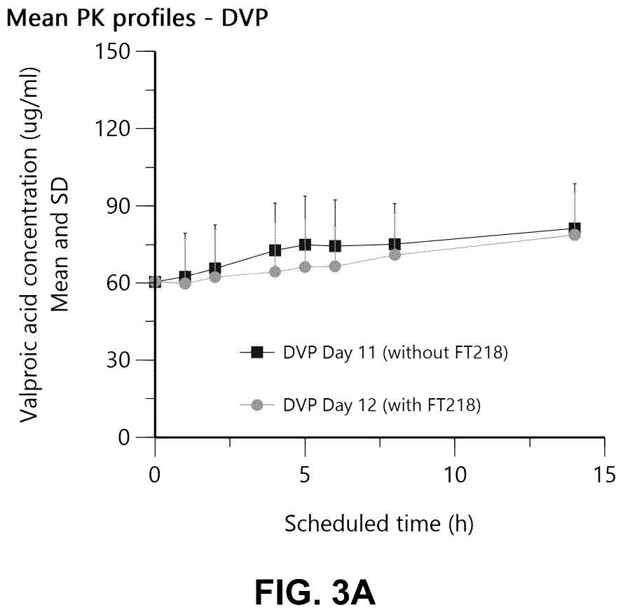

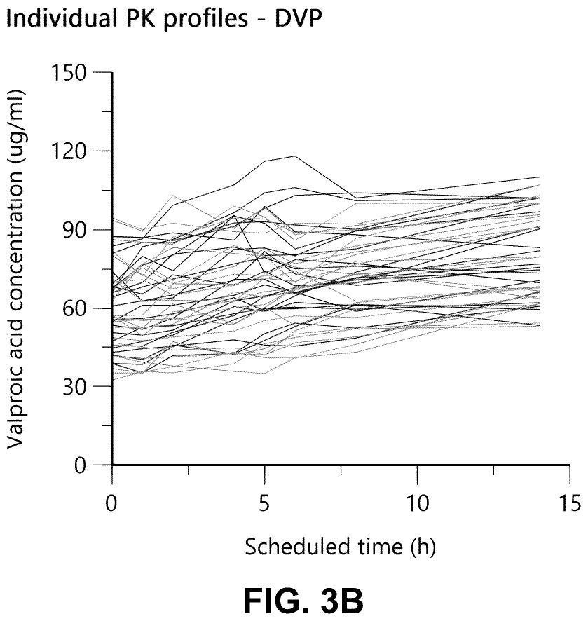

In yet another embodiment, divalproex sodium yields a plasma concentration versus time curve when co-administered with the composition of gamma-hydroxybutyrate once nightly at a strength of 6 g substantially as depicted in FIG. 3A and 3B.

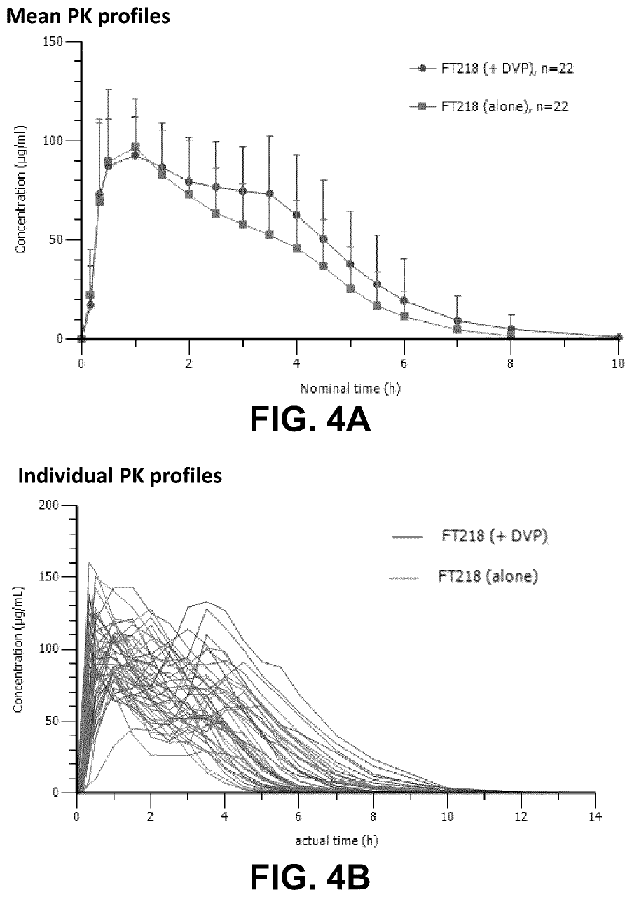

Another embodiment of the composition of gamma-hydroxybutyrate yields a plasma concentration versus time curve when administered once nightly at a strength of 6 g concomitantly with divalproex sodium substantially as depicted in FIG. 4.

Formulations that achieve this improved bioavailability when co-administered with divalproex sodium may be described using several different pharmacokinetic parameters. Compositions of gamma-hydroxybutyrate administered once nightly concomitantly with divalproex sodium may achieve a relative bioavailability of greater than 80%, 85%, 90%, or 95% when compared to an equal dose of the gamma-hydroxybutyrate composition administered without divalproex sodium.

In an embodiment, the AUC_{inf} for the gamma-hydroxybutyrate composition administered concomitantly with divalproex sodium may be substantially similar to the AUC_{inf} when the same the same dosage of the composition is administered alone. For example, when the gamma-hydroxybutyrate composition is co-administered with divalproex sodium, it achieves a mean AUC_{inf} that is from 80% to 125%, from 80% to 100%, from 90% to 100%, from 90% to 115%, from 100% to 120%, or from 110% to 125% of the mean AUC_{inf} provided by an equal dose of the gamma-hydroxybutyrate composition administered without divalproex sodium. In at least one example, when the gamma-hydroxybutyrate composition is co-administered with divalproex sodium, it achieves a mean AUC_{inf} that is about 117% of the mean AUC_{inf} provided by an equal dose of the gamma-hydroxybutyrate composition administered without divalproex sodium. This may be seen by comparing the release profiles and pharmacokinetic profiles in Examples 1-6.

An embodiment of the composition of gamma-hydroxybutyrate includes immediate release and modified release portions, where a 6 g dose of the formulation, when administered with divalproex sodium, may achieve a mean AUC_{inf} of greater than 220 hr*μg/mL. In particular, a 6 g dose of a composition of gamma-hydroxybutyrate co-administered with divalproex may achieve a mean AUC_{inf} of greater than 250 hr*μg/mL, 300 hr*μg/mL, 350 hr*μg/mL, 400 hr*μg/mL, 450 hr*μg/mL, 500 hr*μg/mL, or less than 512 hr*μg/mL. For example, a 6 g dose of the composition co-administered with divalproex sodium may have a mean AUC_{inf} of about 366 hr*μg/mL. In addition, the 6 g dose of the composition may be administered once daily, in the morning or the evening.

The AUC_{inf} for the composition administered with DVP is within the bioequivalent range of the same composition administered alone. In various examples, the 90% confidence interval of the geometric mean ratio of the AUC_{inf} for the composition and DVP/composition (alone) is about 111 to about 122. In at least one example, the ratio is about 116.52.

In an embodiment, the Cₘₐₓ for the gamma-hydroxybutyrate composition administered concomitantly with divalproex sodium may be substantially similar to the Cₘₐₓ when the same dosage of the gamma-hydroxybutyrate composition is administered alone. In an example, when the gamma-hydroxybutyrate composition is co-administered with divalproex sodium, it achieves a mean Cₘₐₓ that is from 80% to 125%, from 80% to 100%, from 90% to 100%, from 95% to 110%, from 100% to 120%, or from 110% to 125% of the mean Cₘₐₓ provided by an equal dose of the gamma-hydroxybutyrate composition administered without divalproex sodium. In at least one example, when the gamma-hydroxybutyrate composition is co-administered with divalproex sodium, it achieves a mean Cₘₐₓ that is about 98% of the mean Cₘₐₓ provided by an equal dose of the gamma-hydroxybutyrate composition administered without divalproex sodium. This may be seen by comparing the release profiles and pharmacokinetic profiles in Examples 1-6.

An embodiment of the composition of gamma-hydroxybutyrate includes immediate release and modified release portions, where a 6 g dose of the formulation, when administered with divalproex sodium, may achieve a mean Cₘₐₓ of greater than 59 μg/mL. For example, a 6 g dose of the formulation, when co-administered with divalproex sodium, may achieve a mean Cₘₐₓ of greater than 65μg/mL, 70 μg/mL, 75 μg/mL, 80 μg/mL, 85 μg/mL, 90 μg/mL, 95 μg/mL, or less than 97 μg/mL. For example, a 6 g dose of the composition co-administered with divalproex sodium has a mean Cₘₐₓ of about 78 μg/mL. In addition, the 6 g dose of the composition may be administered once daily, in the morning or the evening.

The Cₘₐₓ for the composition administered with DVP is within the bioequivalent range of the same composition administered alone. In various examples, the 90% confidence interval of the geometric mean ratio of the Cₘₐₓ for the composition and DVP/composition (alone) is about 91 to about 106. In at least one example, the ratio is about 98.46.

In an embodiment, the AUC₀₋ₗₐₛₜ for the gamma-hydroxybutyrate composition administered concomitantly with divalproex sodium may be substantially similar to the AUC₀₋ₗₐₛₜ when the same the same dosage of the gamma-hydroxybutyrate composition is administered alone. In some examples, when the gamma-hydroxybutyrate composition is co-administered with divalproex sodium, it achieves a mean AUC₀₋ₗₐₛₜ that is from 80% to 125%, from 80% to 100%, from 90% to 100%, from 95% to 110%, or from 100% to 125% of the mean AUC₀₋ₗₐₛₜ provided by an equal dose of the gamma-hydroxybutyrate composition administered without divalproex sodium. In at least one example, when the gamma-hydroxybutyrate composition is co-administered with divalproex sodium, it achieves a mean AUC₀₋ₗₐₛₜ that is about 117% of the mean AUC₀₋ₗₐₛₜ provided by an equal dose of the gamma-hydroxybutyrate composition administered without divalproex sodium. This may be seen by comparing the release profiles and pharmacokinetic profiles in Examples 1-6.

In various embodiments, a 6 g dose of the composition of gamma-hydroxybutyrate may be characterized as having been shown to achieve a mean AUC₀₋ₗₐₛₜ of greater than 220 hr*μg/mL, 250 hr*μg/mL, 300 hr*μg/mL, 350 hr*μg/mL, 400 hr*μg/mL, 450 hr*μg/mL, 500 hr*μg/mL, or less than 512 hr*μg/mL when co-administered with divalproex sodium. For example, a 6 g dose of the composition co-administered with divalproex sodium may have a mean AUC₀₋ₗₐₛₜ of about 366 hr*μg/mL. In addition, the 6 g dose of the composition may be administered once daily, in the morning or the evening.

The AUC₀₋ₗₐₛₜ for the composition administered with DVP is within the bioequivalent range of the same composition administered alone. In various examples, the 90% confidence interval of the geometric mean ratio of the AUC₀₋ₗₐₛₜ for the composition and DVP/composition (alone) is about 111 to about 122. In at least one example, the ratio is about 116.67.

In an embodiment, the gamma-hydroxybutyrate composition administered concomitantly with divalproex sodium may provide mean blood concentrations (μg/ml) at 8 hours substantially similar to that of the same dosage of the gamma-hydroxybutyrate composition when administered alone. In an example, when the gamma-hydroxybutyrate composition is co-administered with divalproex sodium, it achieves a mean C8h that is from 40% to 60%, from 60% to 80%, from 80% to 125%, from 80% to 100%, from 90% to 100%, from 90% to 115%, or from 100% to 125% of the mean Can provided by an equal dose of the gamma-hydroxybutyrate composition administered without divalproex sodium. This may be seen by comparing the release profiles and pharmacokinetic profiles in Examples 1-6.

In various embodiments, a 6 g dose of the composition of gamma-hydroxybutyrate may be characterized as having been shown to achieve a mean C₈ₕ Of greater than 1 μg/mL, 2 μg/mL, 4 μg/mL, 6 μg/mL, 8 μg/mL, 10 μg/mL, 12 μg/mL, 14 μg/mL, 16 μg/mL, 18 μg/mL, or 20 μg/mL when co-administered with divalproex sodium. For example, a 6 g dose of the composition co-administered with divalproex sodium has a mean C₈ₕ of about 9.8 μg/mL. In addition, the 6 g dose of the composition may be administered once daily, in the morning or the evening.

In an embodiment, the Tₘₐₓ for the gamma-hydroxybutyrate composition administered concomitantly with divalproex sodium may be substantially similar to the Tₘₐₓ when the same dosage of the gamma-hydroxybutyrate composition is administered alone. In some examples, when the gamma-hydroxybutyrate composition is co-administered with divalproex sodium, it achieves a mean Tₘₐₓ that is from 60% to 80%, from 70% to 90%, 80% to 125%, from 80% to 100%, from 90% to 100%, from 90% to 115%, or from 100% to 125% of the mean Tₘₐₓ provided by an equal dose of the gamma-hydroxybutyrate composition administered without divalproex sodium. This may be seen by comparing the release profiles and pharmacokinetic profiles in Examples 1-6.

The compositions of gamma-hydroxybutyrate may also be defined based on the time required to reach maximum blood concentration of gamma-hydroxybutyrate. Thus, in additional embodiments, the composition of gamma-hydroxybutyrate may achieve a mean Tₘₐₓ of 0.3 to 3.5 hours. In various embodiments, the composition of gamma-hydroxybutyrate may achieve a mean Tₘₐₓ of about 0.5, 0.75 hours, 1.0 hour, 1.5 hours, 2.0 hours, 2.25 hours, 2.5 hours, 3 hours, or 3.5 hours when co-administered with divalproex sodium. For example, a 6 g dose of the composition co-administered with divalproex sodium may have a median Tₘₐₓ of about 2 hours. In addition, the 6 g dose of the composition may be administered once daily, in the morning or the evening.

In an embodiment, the composition provides an AUC_{inf} that is dose proportional when co-administered with divalproex sodium. In an embodiment, the composition provides a Cₘₐₓ that is dose proportional when co-administered with divalproex sodium. In various embodiments, the composition exhibits pharmacokinetics that is dose proportional when administered once daily, concomitant with divalproex sodium. For example, the composition provides a Cₘₐₓ that is dose proportional across once daily doses of 4.5 g, 7.5 g, 6 g, and 9 g, such that, the Cₘₐₓ of a 6 g dose is proportional to the Cₘₐₓ of a 9 g dose of the composition. The composition may exhibit predictable increases in plasma levels with increasing doses, consistent with the PK profile desired for a once-nightly sodium oxybate formulation.

### Structural Embodiments

The compositions of gamma-hydroxybutyrate may be provided in any dosage form that is suitable for oral administration, including tablets, capsules, liquids, orally dissolving tablets, and the like. In one embodiment, they are provided as dry particulate formulations (i.e. granules, powders, coated particles, microparticles, pellets, microspheres, etc.), in a sachet or other suitable discreet packaging units. A particulate formulation will be mixed with tap water shortly before administration. In one embodiment, the composition may be mixed with 50 mL water prior to administration. In another embodiment, the composition is an oral pharmaceutical composition.

In various embodiments, the composition includes gamma-hydroxybutyrate present in a unit dose of at least 4.5 g, at least 6.0 g, at least 7.5 g, or at least 9.0 g. In various embodiments, the composition includes gamma-hydroxybutyrate present in a unit dose of more than 4.5 g, more than 6.0 g, more than 7.5 g, or more than 9.0 g. In one example, the formulation includes 6 g gamma-hydroxybutyrate. In another example, the formulation includes 7.5 g gamma-hydroxybutyrate. In yet another example, the formulation includes 9 g gamma-hydroxybutyrate. In some embodiments, the dosage of gamma-hydroxybutyrate may be sufficient to administer the composition once daily.

In one embodiment, the formulation comprises immediate release and modified release portions, wherein: (a) the modified release portion comprises coated microparticles of gamma-hydroxybutyrate; and (b) the ratio of gamma-hydroxybutyrate in the immediate release portion and the modified release portion is from 10/90 to 65/35.

In one embodiment, the formulation comprises immediate release and modified release portions, wherein: (a) the modified release portion comprises coated microparticles of gamma-hydroxybutyrate; and (b) the ratio of gamma-hydroxybutyrate in the immediate release portion and the modified release portion is from 40/60 to 60/40.

In another embodiment, the formulation comprises immediate release and modified release portions, wherein: (a) the modified release portion comprises coated microparticles of gamma-hydroxybutyrate; (b) the coating of said modified release particles of gamma-hydroxybutyrate comprises a polymer carrying free carboxylic groups and a hydrophobic compound having a melting point equal or greater than 40° C.; and (c) the ratio of gamma-hydroxybutyrate in the immediate release portion and the modified release portion is from 10/90 to 65/35 or 40/60 to 60/40.

In an embodiment, the composition of gamma-hydroxybutyrate may include immediate release and modified release portions, a suspending or viscosifying agent, and an acidifying agent, wherein: (a) the modified release portion comprises coated particles of gamma-hydroxybutyrate; (b) the coating comprises a polymer carrying free carboxylic groups and a hydrophobic compound having a melting point equal or greater than 40° C.; and (c) the ratio of gamma-hydroxybutyrate in the immediate release portion and the modified release portion is from 10/90 to 65/35.

In another embodiment, the formulation comprises immediate release and modified release portions, wherein: (a) the modified release portion comprises coated microparticles of gamma-hydroxybutyrate; (b) the coating of said modified release particles of gamma-hydroxybutyrate comprises a polymer carrying free carboxylic groups and a hydrophobic compound having a melting point equal or greater than 40° C.; (c) the weight ratio of the hydrophobic compound to the polymer carrying free carboxylic groups is from 0.4 to 4; (d) the ratio of gamma-hydroxybutyrate in the immediate release portion and the modified release portion is from 10/90 to 65/35 or 40/60 to 60/40; and (e) the film coating is from 10 to 50% of the weight of the microparticles.

In another embodiment the formulation comprises immediate release and modified release portions, wherein: (a) the modified release portion comprises coated particles of gamma-hydroxybutyrate; (b) the coating of said modified release particles of gamma-hydroxybutyrate comprises a polymer carrying free carboxylic groups having a pH trigger of from 5.5 to 6.97 and a hydrophobic compound having a melting point equal or greater than 40° C.; (c) the weight ratio of the hydrophobic compound to the polymer carrying free carboxylic groups is from 0.4 to 4; (d) the ratio of gamma-hydroxybutyrate in the immediate release portion and the modified release portion is from 10/90 to 65/35 or 40/60 to 60/40; and (e) the coating is from 10 to 50% of the weight of the particles.

In an embodiment, the polymer carrying free carboxylic groups comprises from 100% poly (methacrylic acid, ethyl acrylate) 1:1 and 0% poly (methacrylic acid, methylmethacrylate) 1:2 to 2% poly (methacrylic acid, ethyl acrylate) 1:1 and 98% poly (methacrylic acid, methylmethacrylate) 1:2; and the hydrophobic compound comprises hydrogenated vegetable oil.

In an embodiment, the formulation includes excipients to improve the viscosity and the pourability of the mixture of the particulate formulation with tap water. As such, the particulate formulation comprises, besides the immediate release and modified release particles of gamma-hydroxybutyrate, one or more suspending or viscosifying agents or lubricants.

Suspending or viscosifying agents may be chosen from the group consisting of xanthan gum, medium viscosity sodium carboxymethyl cellulose, mixtures of microcrystalline cellulose and sodium carboxymethyl cellulose, mixtures of microcrystalline cellulose and guar gum, medium viscosity hydroxyethyl cellulose, agar, sodium alginate, mixtures of sodium alginate and calcium alginate, gellan gum, carrageenan gum grade iota, kappa or lambda, and medium viscosity hydroxypropylmethyl cellulose.

Medium viscosity sodium carboxymethyl cellulose corresponds to grade of sodium carboxymethyl cellulose whose viscosity, for a 2% solution in water at 25° C., is greater than 200 mPa·s and lower than 3100 mPa·s.

Medium viscosity hydroxyethyl cellulose corresponds to a grade of hydroxyethyl cellulose whose viscosity, for a 2% solution in water at 25° C., is greater than 250 mPa·s and lower than 6500 mPa·s. Medium viscosity hydroxypropylmethyl cellulose corresponds to a grade of hydroxypropylmethyl cellulose whose viscosity, for a 2% solution in water at 20° C., is greater than 80 mPa·s. and lower than 3800 mPa·s.

In one embodiment, the suspending or viscosifying agents are xanthan gum, especially Xantural 75™from Kelco, hydroxyethylcellulose, especially Natrosol 250M™ from Ashland, Kappa carrageenan gum, especially Gelcarin PH812™ from FMC Biopolymer, and lambda carrageenan gum, especially Viscarin PH209™ from FMC Biopolymer.

In an embodiment, the composition of gamma-hydroxybutyrate comprises from 1 to 15% of viscosifying or suspending agents. In other embodiments, the composition of gamma-hydroxybutyrate comprises viscosifying or suspending agents in an amount from 2 to 10%, from 2 to 5%, or from 2 to 3% of the formulation.

In an embodiment, the composition of gamma-hydroxybutyrate is in the form of a powder that is intended to be dispersed in water prior to administration and further comprises from 1 to 15% of a suspending or viscosifying agent selected from a mixture of xanthan gum, carrageenan gum and hydroxyethylcellulose or xanthan gum and carrageenan gum.

In an embodiment, the composition of gamma-hydroxybutyrate is in the form of a powder that is intended to be dispersed in water prior to administration and further comprises: from 1.2 to 15% of an acidifying agent selected from malic acid and tartaric acid; and from 1 to 15% of a suspending or viscosifying agent selected from a mixture of xanthan gum, carrageenan gum and hydroxyethylcellulose or xanthan gum and carrageenan gum.

In one embodiment, the composition of gamma-hydroxybutyrate comprises about 1% of lambda carrageenan gum or Viscarin PH209™, about 1% of medium viscosity grade of hydroxyethyl cellulose or Natrosol 250M™, and about 0.7% of xanthan gum or Xantural 75™. For a 4.5 g dose unit, these percentages will typically equate to about 50 mg xanthan gum (Xantural 75TM), about 75 mg carragenan gum (Viscarin PH209TM), and about 75 mg hydroxyethylcellulose (Natrasol 250MTM).

Alternative packages of viscosifying or suspending agents, for a 4.5 g dose, include about 50 mg xanthan gum (Xantural 75TM) and about 100 mg carragenan gum (Gelcarin PH812TM), or about 50 mg xanthan gum (Xantural 75TM), about 75 mg hydroxyethylcellulose (Natrasol 250MTM), and about 75 mg carragenan gum (Viscarin PH109TM).

In an embodiment, the composition of gamma-hydroxybutyrate further comprises a lubricant or a glidant, besides the immediate release and modified release particles of gamma-hydroxybutyrate. In various embodiments, the lubricants and glidants are chosen from the group consisting of salts of stearic acid, in particular magnesium stearate, calcium stearate or zinc stearate, esters of stearic acid, in particular glyceryl monostearate or glyceryl palmitostearate, stearic acid, glycerol behenate, sodium stearyl fumarate, talc, and colloidal silicon dioxide. In one embodiment, the lubricant or glidant is magnesium stearate. The lubricant or glidant may be used in the particulate formulation in an amount of from 0.1 to 5%. In one embodiment, the amount of lubricant or glidant is about 0.5%. For example, the composition of gamma-hydroxybutyrate may include about 0.5% of magnesium stearate.

A composition of gamma-hydroxybutyrate may further include an acidifying agent. The acidifying agent helps to ensure that the release profile of the formulation in 0.1N HCl will remain substantially unchanged for at least 15 minutes after mixing, which is approximately the maximum length of time a patient might require before consuming the dose after mixing the formulation with tap water.

In one embodiment, the formulation is a powder, and further comprising an acidifying agent and a suspending or viscosifying agent in the weight percentages recited herein.

The acidifying agents may be chosen from the group consisting of malic acid, citric acid, tartaric acid, adipic acid, boric acid, maleic acid, phosphoric acid, ascorbic acid, oleic acid, capric acid, caprylic acid, and benzoic acid. In various embodiments, the acidifying agent is present in the formulation from 1.2 to 15%, from 1.2 to 10%, or from 1.2 to 5%. In one embodiment, the acidifying agents are tartaric acid and malic acid. In another embodiment, the acidifying agent is malic acid.

When tartaric acid is employed, it may be employed in an amount of from 1 to 10%, from 2.5 to 7.5%, or about 5%. In various embodiments, the amount of malic acid in the composition of gamma-hydroxybutyrate is from 1.2 to 15%, from 1.2 to 10%, from 1.2 to 5%, or from 1.6% or 3.2%. In one embodiment, the amount of malic acid in the composition of gamma hydroxybutyrate is about 1.6%.

The composition of gamma-hydroxybutyrate includes an immediate release portion and a modified release portion of gamma-hydroxybutyrate, and in an embodiment, the formulation is a particulate formulation that includes a plurality of immediate release gamma-hydroxybutyrate particles and a plurality of modified release gamma-hydroxybutyrate particles. The molar ratio of gamma-hydroxybutyrate in the immediate release and modified release portions ranges from 0.11:1 to 1.86:1, from 0.17:1 to 1.5:1, from 0.25:1 to 1.22:1, from 0.33:1 to 1.22:1, from 0.42:1 to 1.22:1, from 0.53:1 to 1.22:1, from 0.66:1 to 1.22:1, from 0.66:1 to 1.5:1, from 0.8:1 to 1.22:1. In one embodiment, the molar ratio of gamma-hydroxybutyrate in the immediate release and modified release portions is about 1:1. The molar percentage of gamma-hydroxybutyrate in the immediate release portion relative to the total of gamma-hydroxybutyrate in the formulation ranges from 10% to 65%, from 15 to 60%, from 20 to 55%, from 25 to 55%, from 30 to 55%, from 35 to 55%, from 40 to 55%, from 40 to 60%, or from 45 to 55%. In one embodiment, the molar percentage of gamma-hydroxybutyrate in the immediate release portion relative to the total of gamma-hydroxybutyrate in the formulation ranges from 40% to 60%. In an embodiment, the molar percentage of the gamma-hydroxybutyrate in the immediate release portion relative to the total of gamma-hydroxybutyrate in the formulation is about 50%. The molar percentage of gamma-hydroxybutyrate in the modified release portion relative to the total of gamma-hydroxybutyrate in the formulation ranges from 90% to 35%, from 85 to 40%, from 80 to 45%, from 75 to 45%, from 70 to 45%, from 65 to 45%, from 60 to 45%, from 60 to 40%, or from 55 to 45%. In an embodiment, the molar percentage of gamma-hydroxybutyrate in the modified release portion relative to the total of gamma-hydroxybutyrate in the formulation ranges from 60% to 40%. In one embodiment, the molar ratio of the gamma-hydroxybutyrate in the modified release portion relative to the total of gamma-hydroxybutyrate in the formulation is about 50%. The weight percentage of the IR microparticles relative to the total weight of IR microparticles and MR microparticles ranges from 7.2% to 58.2%, from 11.0% to 52.9%, from 14.9% to 47.8%, from 18.9% to 47.8%, from 23.1% to 47.8%, from 27.4% to 47.8%, from 31.8% to 47.8%, from 31.8% to 52.9%, or from 36.4% to 47.8%. In other embodiments, the weight percentage of the IR microparticles relative to the total weight of IR microparticles and MR microparticles ranges from 5.9% to 63.2%, from 9.1% to 58.1%, from 12.4% to 53.1%, from 19.9% to 53.1%, from 19.6% to 53.1%, from 23.4% to 53.1%, from 27.4% to 53.1%, or from 27.4% to 58.1%. In one embodiment, the weight percentage of the IR microparticles relative to the total weight of IR microparticles and MR microparticles ranges from 31,7% to 53.1%.

In an embodiment, the finished formulation comprises 50% of its sodium oxybate content in immediate-release particles consisting of 80.75% w/w of sodium oxybate, 4.25% w/w of Povidone K30 and 15% of microcrystalline cellulose spheres with a volume mean diameter of about 95 microns to 450 microns and 50% of its sodium oxybate content in modified release particles consisting of 10.5% w/w of microcrystalline cellulose spheres with a volume mean diameter of about 95 microns to about 450 microns, layered with 56.5% w/w of sodium oxybate mixed with 3% w/w of Povidone™ K30 and finally coated with a coating composition consisting of 18% w/w of hydrogenated vegetable oil (Lubritab™ or equivalent), 4% of methacrylic acid copolymer type C (Eudragit™ L100-55 or equivalent) and 8% of methacrylic acid copolymer type B (Eudragit™ S100 or equivalent).

In an embodiment, the finished formulation comprises 50% of its sodium oxybate content in immediate-release particles consisting of 80.75% w/w of sodium oxybate, 4.25% w/w of Povidone K30 and 15% of microcrystalline cellulose spheres with a volume mean diameter of about 95 microns to 170 microns and 50% of its sodium oxybate content in modified release particles consisting of 10.5% w/w of microcrystalline cellulose spheres with a volume mean diameter of about 95 microns to about 170 microns, layered with 56.5% w/w of sodium oxybate mixed with 3% w/w of Povidone™ K30 and finally coated with a coating composition consisting of 18% w/w of hydrogenated vegetable oil (Lubritab™ or equivalent), 4% of methacrylic acid copolymer type C (Eudragit™ L100-55 or equivalent) and 8% of methacrylic acid copolymer type B (Eudragit™ S100 or equivalent).

In an embodiment, the finished formulation comprises 50% of its sodium oxybate content in immediate-release particles consisting of 80.75% w/w of sodium oxybate, 4.25% w/w of Povidone K30 and 15% of microcrystalline cellulose spheres with a volume mean diameter of about 95 microns to about 450 microns and 50% of its sodium oxybate content in modified release particles consisting of 11.3% w/w of microcrystalline cellulose spheres with a volume mean diameter of about 95 microns to about 450 microns, layered with 60.5% w/w of sodium oxybate mixed with 3.2% w/w of Povidone™ K30 and finally coated with a coating composition consisting of 15% w/w of hydrogenated vegetable oil (Lubritab™ or equivalent), 0.75% of methacrylic acid copolymer type C (Eudragit™ L100-55 or equivalent) and 9.25% of methacrylic acid copolymer type B (Eudragit™ S100 or equivalent).

In an embodiment, the finished formulation comprises 50% of its sodium oxybate content in immediate-release particles consisting of 80.75% w/w of sodium oxybate, 4.25% w/w of Povidone™ K30 and 15% of microcrystalline cellulose spheres with a volume mean diameter of about 95 microns to about 170 microns and 50% of its sodium oxybate content in modified release particles consisting of 11.3% w/w of microcrystalline cellulose spheres with a volume mean diameter of about 95 microns to about 170 microns, layered with 60.5% w/w of sodium oxybate mixed with 3.2% w/w of Povidone™ K30 and finally coated with a coating composition consisting of 15% w/w of hydrogenated vegetable oil (Lubritab™ or equivalent), 0.75% of methacrylic acid copolymer type C (Eudragit™ L100-55 or equivalent) and 9.25% of methacrylic acid copolymer type B (Eudragit™ S100 or equivalent).

In an embodiment, the finished formulation comprises 50% of its gamma-hydroxybutyrate content in immediate-release particles consisting of 80.75% w/w of potassium salt of gamma-hydroxybutyric acid, 4.25% w/w of Povidone K30 and 15% of microcrystalline cellulose spheres with a volume mean diameter of about 95 microns to about 450 microns and 50% of its gamma-hydroxybutyrate content in modified release particles consisting of 10.5% w/w of microcrystalline cellulose spheres with a volume mean diameter of about 95 microns to about 450 microns, layered with 56.5% w/w of sodium oxybate mixed with 3% w/w of Povidone™ K30 and finally coated with a coating composition consisting of 18% w/w of hydrogenated vegetable oil (Lubritab™ or equivalent), 4% of methacrylic acid copolymer type C (Eudragit™ L100-55or equivalent) and 8% of methacrylic acid copolymer type B (Eudragit™ S100 or equivalent).

In an embodiment, the finished formulation comprises 50% of its gamma-hydroxybutyrate content in immediate-release particles consisting of 80.75% w/w of potassium salt of gamma-hydroxybutyric acid, 4.25% w/w of Povidone K30 and 15% of microcrystalline cellulose spheres with a volume mean diameter of about 95 microns to about 170 microns and 50% of its gamma-hydroxybutyrate content in modified release particles consisting of 10.5% w/w of microcrystalline cellulose spheres with a volume mean diameter of about 95 microns to about 170 microns, layered with 56.5% w/w of sodium oxybate mixed with 3% w/w of Povidone™ K30 and finally coated with a coating composition consisting of 18% w/w of hydrogenated vegetable oil (Lubritab™ or equivalent), 4% of methacrylic acid copolymer type C (Eudragit™ L100-55or equivalent) and 8% of methacrylic acid copolymer type B (Eudragit™ S100 or equivalent).

In an embodiment, the finished formulation comprises 16.7% of its gamma-hydroxybutyrate content in immediate-release particles consisting of 80.75% w/w of potassium salt of gamma-hydroxybutyric acid, 4.25% w/w of Povidone K30 and 15% of microcrystalline cellulose spheres with a volume mean diameter of about 95 microns to about 450 microns, 16.7% of its gamma-hydroxybutyrate content in immediate-release particles consisting of 80.75% w/w of magnesium salt of gamma-hydroxybutyric acid, 4.25% w/w of Povidone K30 and 15% of microcrystalline cellulose spheres with a volume mean diameter of about 95 microns to about 450 microns, 16.7% of its gamma-hydroxybutyrate content in immediate-release particles consisting of 80.75% w/w of calcium salt of gamma-hydroxybutyric acid, 4.25% w/w of Povidone K30 and 15% of microcrystalline cellulose spheres with a volume mean diameter of about 95 microns to about 450 microns and 50% of its gamma-hydroxybutyrate content in modified release particles consisting of 10.5% w/w of microcrystalline cellulose spheres with a volume mean diameter of about 95 microns to about 450 microns, layered with 56.5% w/w of sodium oxybate mixed with 3% w/w of Povidone™ K30 and finally coated with a coating composition consisting of 18% w/w of hydrogenated vegetable oil (Lubritab™ or equivalent), 4% of methacrylic acid copolymer type C (Eudragit™ L100-55or equivalent) and 8% of methacrylic acid copolymer type B (Eudragit™ S100 or equivalent).

In an embodiment, the finished formulation comprises 16.7% of its gamma-hydroxybutyrate content in immediate-release particles consisting of 80.75% w/w of potassium salt of gamma-hydroxybutyric acid, 4.25% w/w of Povidone K30 and 15% of microcrystalline cellulose spheres with a volume mean diameter of about 95 microns to about 170 microns, 16.7% of its gamma-hydroxybutyrate content in immediate-release particles consisting of 80.75% w/w of magnesium salt of gamma-hydroxybutyric acid, 4.25% w/w of Povidone K30 and 15% of microcrystalline cellulose spheres with a volume mean diameter of about 95 microns to about 170 microns, 16.7% of its gamma-hydroxybutyrate content in immediate-release particles consisting of 80.75% w/w of calcium salt of gamma-hydroxybutyric acid, 4.25% w/w of Povidone K30 and 15% of microcrystalline cellulose spheres with a volume mean diameter of about 95 microns to about 170 microns and 50% of its gamma-hydroxybutyrate content in modified release particles consisting of 10.5% w/w of microcrystalline cellulose spheres with a volume mean diameter of about 95 microns to about 170 microns, layered with 56.5% w/w of sodium oxybate mixed with 3% w/w of Povidone™ K30 and finally coated with a coating composition consisting of 18% w/w of hydrogenated vegetable oil (Lubritab™ or equivalent), 4% of methacrylic acid copolymer type C (Eudragit™ L100-55or equivalent) and 8% of methacrylic acid copolymer type B (Eudragit™ S100 or equivalent).

In an embodiment, the finished formulation comprises 50% of its gamma-hydroxybutyrate content in immediate-release particles consisting of 80.75% w/w of potassium salt of gamma-hydroxybutyric acid, 4.25% w/w of Povidone K30 and 15% of microcrystalline cellulose spheres with a volume mean diameter of about 95 microns to about 450 microns and 50% of its gamma-hydroxybutyrate content in modified release particles consisting of 10.5% w/w of microcrystalline cellulose spheres with a volume mean diameter of about 95 microns to about 450 microns, layered with 56.5% w/w of calcium salt of gamma-hydroxybutyric acid mixed with 3% w/w of Povidone™ K30 and finally coated with a coating composition consisting of 18% w/w of hydrogenated vegetable oil (Lubritab™ or equivalent), 4% of methacrylic acid copolymer type C (Eudragit™ L100-55 or equivalent) and 8% of methacrylic acid copolymer type B (Eudragit™ S100 or equivalent).

In an embodiment, the finished formulation comprises 50% of its gamma-hydroxybutyrate content in immediate-release particles consisting of 80.75% w/w of potassium salt of gamma-hydroxybutyric acid, 4.25% w/w of Povidone K30 and 15% of microcrystalline cellulose spheres with a volume mean diameter of about 95 microns to about 170 microns and 50% of its gamma-hydroxybutyrate content in modified release particles consisting of 10.5% w/w of microcrystalline cellulose spheres with a volume mean diameter of about 95 microns to about 170 microns, layered with 56.5% w/w of calcium salt of gamma-hydroxybutyric acid mixed with 3% w/w of Povidone™ K30 and finally coated with a coating composition consisting of 18% w/w of hydrogenated vegetable oil (Lubritab™ or equivalent), 4% of methacrylic acid copolymer type C (Eudragit™ L100-55 or equivalent) and 8% of methacrylic acid copolymer type B (Eudragit™ S100 or equivalent).

### Other Characteristics of Immediate Release Portion

The immediate release portion of the formulation can take any form capable of achieving an immediate release of the gamma-hydroxybutyrate when ingested. For example, when the formulation is a particulate formulation, the formulation can include unmodified “raw” gamma-hydroxybutyrate, rapidly dissolving gamma-hydroxybutyrate granules, particles or microparticles comprised of a core covered by a gamma-hydroxybutyrate loaded layer containing a binder such as povidone.

The IR granules or particles of gamma-hydroxybutyrate may be made using any manufacturing process suitable to produce the required particles, including:
agglomeration of the gamma-hydroxybutyrate sprayed in the molten state, such as the Glatt ProCell™ technique,
extrusion and spheronization of the gamma-hydroxybutyrate, optionally with one or more physiologically acceptable excipients,
wet granulation of the gamma-hydroxybutyrate, optionally with one or more physiologically acceptable excipients,
compacting of the gamma-hydroxybutyrate, optionally with one or more physiologically acceptable excipients,
granulation and spheronization of the gamma-hydroxybutyrate, optionally with one or more physiologically acceptable excipients, the spheronization being carried out for example in a fluidized bed apparatus equipped with a rotor, in particular using the Glatt CPS™ technique,
spraying of the gamma-hydroxybutyrate, optionally with one or more physiologically acceptable excipients, for example in a fluidized bed type apparatus equipped with zig-zag filter, in particular using the Glatt MicroPx™ technique, or
spraying, for example in a fluidized bed apparatus optionally equipped with a partition tube or Wurster tube, the gamma-hydroxybutyrate, optionally with one or more physiologically acceptable excipients, in dispersion or in solution in an aqueous or organic solvent on a core.

The immediate release portion of the formulation is in the form of microparticles comprising the immediate release gamma-hydroxybutyrate and optional pharmaceutically acceptable excipients. In an embodiment, the immediate release microparticles of gamma-hydroxybutyrate have a volume mean diameter D(4,3) of from 10 to 1000 microns. In other embodiments, the immediate release microparticles of gamma-hydroxybutyrate have a volume mean diameter D(4,3) of from 95 to 600 microns. In additional embodiments, the immediate release microparticles of gamma-hydroxybutyrate have a volume mean diameter D(4,3) of from 150 to 400 microns. In one embodiment, their volume mean diameter is about 270 microns.

The immediate release particles of gamma-hydroxybutyrate may include a core and a layer deposited on the core that contains the gamma-hydroxybutyrate. The core may be any particle chosen from the group consisting of:
crystals or spheres of lactose, sucrose (such as Compressuc™ PS from Tereos), microcrystalline cellulose (such as Avicel™ from FMC Biopolymer, Cellet™ from Pharmatrans or Celphere™ from Asahi Kasei), sodium chloride, calcium carbonate (such as Omyapure 35 from Omya), sodium hydrogen carbonate, dicalcium phosphate (such as Dicafos™ AC 92-12 from Budenheim) or tricalcium phosphate (such as Tricafos™ SC93-15 from Budenheim);
composite spheres or granules, for example sugar spheres comprising sucrose and starch (such as Suglets™ from NP Pharm), spheres of calcium carbonate and starch (such as Destab™ 90 S Ultra 250 from Particle Dynamics) or spheres of calcium carbonate and maltodextrin (such as Hubercal™ CCG4100 from Huber).

The core can also comprise other particles of pharmaceutically acceptable excipients such as particles of hydroxypropyl cellulose (such as Klucel™ from Aqualon Hercules), guar gum particles (such as Grinsted™ Guar from Danisco), xanthan particles (such as Xantural™ 180 from CP Kelco).

According to a particular embodiment of the invention, the cores are sugar spheres or microcrystalline cellulose spheres, such as Cellets™ 90, Cellets™ 100 or Cellets™ 127 marketed by Pharmatrans, or also Celphere™ CP 203, Celphere™ CP305, Celphere™ SCP 100. In one embodiment, the core is a microcrystalline cellulose sphere. For example, the core may be a Cellets™ 127 from Pharmatrans.

In various embodiments, the core has a mean volume diameter of about 95 to about 450 microns, about 95 to about 170 microns, or about 140 microns.

The layer deposited onto the core comprises the immediate release gamma-hydroxybutyrate. In an embodiment, the layer also comprises a binder, which may be chosen from the group consisting of:
low molecular weight hydroxypropyl cellulose (such as Klucel™ EF from Aqualon-Hercules), low molecular weight hydroxypropyl methylcellulose (or hypromellose) (such as Methocel™ E3 or E5 from Dow), or low molecular weight methylcellulose (such as Methocel™ A15 from Dow);
low molecular weight polyvinyl pyrrolidone (or povidone) (such as Plasdone™ K29/32 from ISP or Kollidon™ 30 from BASF), vinyl pyrrolidone™: S630 and vinyl acetate copolymer (or copovidone) (such as Plasdone™: from ISP or Kollidon™ VA 64 from BASF);
dextrose, pregelatinized starch, maltodextrin; and mixtures thereof.

Low molecular weight hydroxypropyl cellulose corresponds to grades of hydroxypropyl cellulose having a molecular weight of less than 800,000 g/mol, less than or equal to 400,000 g/mol, or less than or equal to 100,000 g/mol. Low molecular weight hydroxypropyl methylcellulose (or hypromellose) corresponds to grades of hydroxypropyl methylcellulose the solution viscosity of which, for a 2% solution in water and at 20° C., is less than or equal to 1,000 mPa·s, less than or equal to 100 mPa·s, or less than or equal to 15 mPa·s. Low molecular weight polyvinyl pyrrolidone (or povidone) corresponds to grades of polyvinyl pyrrolidone having a molecular weight of less than or equal to 1,000,000 g/mol, less than or equal to 800,000 g/mol, or less than or equal to 100,000 g/mol.

In some embodiments, the binding agent is chosen from low molecular weight polyvinylpyrrolidone or povidone (for example, Plasdone™ K29/32 from ISP), low molecular weight hydroxypropyl cellulose (for example, Klucel™ EF from Aqualon-Hercules), low molecular weight hydroxypropyl methylcellulose or hypromellose (for example, Methocel™ E3 or E5 from Dow) and mixtures thereof.

In one embodiment, the binder is povidone K30 or K29/32, especially Plasdone™ K29/32 from ISP. The binder may be present in an amount of 0 to 80%, 0 to 70%, 0 to 60%, 0 to 50%, 0 to 40%, 0 to 30%, 0 to 25%, 0 to 20%, 0 to 15%, 0 to 10%, or from 1 to 9% of binder based on the total weight of the immediate release coating. In an embodiment, the binder is present in an amount of 5% based on the total weight of the immediate release coating. In one embodiment, the amount of binder is 5% of binder over the total mass of gamma-hydroxybutyrate and binder.

The layer deposited on the core can represent at least 10% by weight, and even greater than 20, 25, 30, 35, 40, 45, 50, 55, 60, 65, 70, 75, 80, 85 or 90% by weight of the total weight of the immediate release particle of gamma-hydroxybutyrate. In one embodiment, the layer deposited on the core represents about 85% of the weight of the immediate release particle of gamma-hydroxybutyrate.

According to an embodiment, the immediate-release particles comprise 80.75% w/w of gamma-hydroxybutyrate, 4.25% w/w of Povidone K30 and 15% of microcrystalline cellulose spheres.

According to another embodiment, the immediate-release particles comprise 80.75% w/w of gamma-hydroxybutyrate, 4.25% w/w of Povidone K30 and 15% of microcrystalline cellulose spheres with a volume mean diameter of about 95 microns to about 450 microns.

According to yet another embodiment, the immediate-release particles comprise 80.75% w/w of gamma-hydroxybutyrate, 4.25% w/w of Povidone K30 and 15% of microcrystalline cellulose spheres with a volume mean diameter of about 95 microns to about 170 microns.

According to an embodiment, the immediate-release particles comprise 80.75% w/w of sodium oxybate, 4.25% w/w of Povidone K30 and 15% of microcrystalline cellulose spheres.

According to another embodiment, the immediate-release particles comprise 80,75% w/w of potassium salt of gamma-hydroxybutyric acid, 4.25% w/w of Povidone K30 and 15% of microcrystalline cellulose spheres.

According to another embodiment, the immediate-release particles comprise 80,75% w/w of calcium salt of gamma-hydroxybutyric acid, 4.25% w/w of Povidone K30 and 15% of microcrystalline cellulose spheres.

According to another embodiment, the immediate-release particles comprise 80,75% w/w of magnesium salt of gamma-hydroxybutyric acid, 4.25% w/w of Povidone K30 and 15% of microcrystalline cellulose spheres.

According to another embodiment, the immediate-release particles are manufactured by dissolving the gamma-hydroxybutyrate and the Povidone K30 in a mixture of water/ethanol 40/60 w/w and spraying the resulting solution onto the surface of the microcrystalline cellulose spheres.

### Other Characteristics of Modified Release Portion

The modified release portion may be any formulation that provides the desired in vitro dissolution profile of gamma-hydroxybutyrate. The modified release portion may include modified release particles, obtained by coating immediate release particles of gamma-hydroxybutyrate with a coating (or coating film) that inhibits the immediate release of the gamma-hydroxybutyrate. In one sub-embodiment the modified release portion comprises particles comprising: (a) an inert core; (b) a coating; and (c) a layer comprising the gamma hydroxybutyrate interposed between the core and the coating.

In an embodiment, the modified release portion comprises a time-dependent release mechanism and a pH-dependent release mechanism.

In an embodiment, the coating film comprises at least one polymer carrying free carboxylic groups, and at least one hydrophobic compound characterized by a melting point equal or greater than 40° C.

The polymer carrying free carboxylic groups may be selected from: (meth)acrylic acid/alkyl (meth)acrylate copolymers or methacrylic acid and methylmethacrylate copolymers or methacrylic acid and ethyl acrylate copolymers or methacrylic acid copolymers type A, B or C, cellulose derivatives carrying free carboxylic groups, cellulose acetate phthalate, cellulose acetate succinate, hydroxypropyl methyl cellulose phthalate, carboxymethylethyl cellulose, cellulose acetate trimellitate, hydroxypropyl methyl cellulose acetate succinate, polyvinyl acetate phthalate, zein, shellac, alginate and mixtures thereof.

In an embodiment, the methacrylic acid copolymers are chosen from the group consisting of poly (methacrylic acid, methyl methacrylate) 1:1 or Eudragit™ L100 or equivalent, poly (methacrylic acid, ethyl acrylate) 1:1 or Eudragit™ L100-55 or equivalent and poly (methacrylic acid, methyl methacrylate) 1:2 or Eudragit™ S100 or equivalent.

In another embodiment the coating comprises a polymer carrying free carboxylic groups wherein the free carboxylic groups are substantially ionized at pH 7.5.

The hydrophobic compound with a melting point equal or greater than 40° C. may be selected from the group consisting of hydrogenated vegetable oils, vegetable waxes, wax yellow, wax white, wax microcrystalline, lanolin, anhydrous milk fat, hard fat suppository base, lauroyl macrogol glycerides, polyglyceryl diisostearate, diesters or triesters of glycerol with a fatty acid, and mixtures thereof.

In various embodiments, the hydrophobic compound with a melting point equal or greater than 40° C. is chosen from the group of following products: hydrogenated cottonseed oil, hydrogenated soybean oil, hydrogenated palm oil, glyceryl behenate, hydrogenated castor oil, candellila wax, tristearin, tripalmitin, trimyristin, yellow wax, hard fat or fat that is useful as suppository bases, anhydrous dairy fats, lanolin, glyceryl palmitostearate, glyceryl stearate, lauryl macrogol glycerides, polyglyceryl diisostearate, diethylene glycol monostearate, ethylene glycol monostearate, omega 3 fatty acids, and mixtures thereof. For example, the hydrophobic compound may include hydrogenated cottonseed oil, hydrogenated soybean oil, hydrogenated palm oil, glyceryl behenate, hydrogenated castor oil, candelilla wax, tristearin, tripalmitin, trimyristin, beeswax, hydrogenated poly-1 decene, carnauba wax, and mixtures thereof.

In practice, and without this being limiting, the hydrophobic compound with a melting point equal or greater than 40° C. may be chosen from the group of products sold under the following trademarks: Dynasan™, Cutina™, Hydrobase™, Dub™, Castorwax™, Croduret™, Compritol™, Sterotex™, Lubritab™ Apifil™, Akofine™, Softisan™, Hydrocote™, Livopol™, Super Hartolan™, MGLA™ Corona™, Protalan™, Akosoft™, Akosol™, Cremao™, Massupol™, Novata™ Suppocire™, Wecobee™, Witepsol™, Lanolin™, Incromega™, Estaram™, Suppoweiss™, Gelucire™, Precirol™, Emulcire™, Plurol diisostearique™, Geleol™, Hydrine™, Monthyle™, Kahlwax™ and mixtures thereof. In an embodiment, the hydrophobic compound with a melting point equal or greater than 40° C. may be chosen from the group of products sold under the following trademarks: Dynasan™MP60, Dynasan™114, Dynasan™116, Dynasan™118, Cutina™ HR, Hydrobase™ 66-68, Dub™ HPH, Compritol™ 888, Sterotex™ NF, Sterotex™ K, Lubritab™, and mixtures thereof.

A particularly suitable coating is composed of a mixture of hydrogenated vegetable oil and a methacrylic acid copolymer. The exact structure and amount of each component, and the amount of coating applied to the particle, controls the release rate and release triggers. Eudragit® methacrylic acid copolymers, namely the methacrylic acid-methyl methacrylate copolymers and the methacrylic acid-ethyl acrylate copolymers, have a pH-dependent solubility: typically, the pH triggering the release of the active ingredient from the microparticles is set by the choice and mixture of appropriate Eudragit® polymers. In the case of gamma hydroxybutyrate modified release microparticles, the theoretical pH triggering the release is from 5.5 to 6.97 or from 5.5 to 6.9. By “pH trigger” is meant the minimum pH above which dissolution of the polymer occurs.

In a particular embodiment, the coating comprises a hydrophobic compound with a melting point equal or greater than 40° C. and a polymer carrying free carboxylic groups are present in a weight ratio from 0.4 to 4, from 0.5 to 4, from 0.6 to 2.5, from 0.67 to 2.5, from 0.6 to 2.33, or from 0.67 to 2.33. In one embodiment, the weight ratio is about 1.5.

A particularly suitable coating is composed of a mixture of hydrogenated vegetable oil and a methacrylic acid copolymer with a theoretical pH triggering the release from 6.5 up to 6.97 in a weight ratio from 0.4 to 4, from 0.5 to 4, from 0.6 to 2.5, from 0.67 to 2.5, from 0.6 to 2.33, or from 0.67 to 2.33. In one embodiment, the weight ratio may be about 1.5.

The modified release particles of gamma-hydroxybutyrate have a volume mean diameter of from 100 to 1200 microns, from 100 to 500 microns, or from 200 to 800 microns. In one embodiment, the modified release particles of gamma-hydroxybutyrate have a volume mean diameter of about 320 microns.

The coating can represent 10 to 50%, 15 to 45%, 20 to 40%, or 25 to 35% by weight of the total weight of the coated modified release particles. In one embodiment, the coating represents 25-30% by weight of the total weight of the modified release particles of gamma-hydroxybutyrate.

In an embodiment, the coating layer of the modified release particles of gamma-hydroxybutyrate is obtained by spraying, in particular in a fluidized bed apparatus, a solution, suspension or dispersion comprising the coating composition as defined previously onto the immediate release particles of gamma-hydroxybutyrate, in particular the immediate release particles of gamma-hydroxybutyrate as previously described. In one embodiment, the coating is formed by spraying in a fluidized bed equipped with a Wurster or partition tube and according to an upward spray orientation or bottom spray a solution of the coating excipients in hot isopropyl alcohol.

According to an embodiment, the modified release particles of gamma-hydroxybutyrate consist of 10.5% w/w of microcrystalline cellulose spheres with a volume mean diameter of about 95 microns to about 450 microns, layered with 56.5% w/w of gamma-hydroxybutyrate mixed with 3% w/w of Povidone™ K30 and finally coated with a coating composition consisting of 18% w/w of hydrogenated vegetable oil (Lubritab™ or equivalent), 4% of methacrylic acid copolymer type C (Eudragit™ L100-55 or equivalent) and 8% of methacrylic acid copolymer type B (Eudragit™ S100 or equivalent), all percentages expressed based on the total weight of the final modified release particles of gamma-hydroxybutyrate.

According to an embodiment, the modified release particles of gamma-hydroxybutyrate consist of 10.5% w/w of microcrystalline cellulose spheres with a volume mean diameter of about 95 microns to about 170 microns, layered with 56.5% w/w of gamma-hydroxybutyrate mixed with 3% w/w of Povidone™ K30 and finally coated with a coating composition consisting of 18% w/w of hydrogenated vegetable oil (Lubritab™ or equivalent), 4% of methacrylic acid copolymer type C (Eudragit™ L100-55 or equivalent) and 8% of methacrylic acid copolymer type B (Eudragit™ S100 or equivalent), all percentages expressed based on the total weight of the final modified release particles of gamma-hydroxybutyrate.

According to an embodiment, the modified release particles of gamma-hydroxybutyrate consist of 10.5% w/w of microcrystalline cellulose spheres with a volume mean diameter of about 95 microns to about 450 microns, layered with 56.5% w/w of sodium oxybate mixed with 3% w/w of Povidone™ K30 and finally coated with a coating composition consisting of 18% w/w of hydrogenated vegetable oil (Lubritab™ or equivalent), 4% of methacrylic acid copolymer type C (Eudragit™ L100-55 or equivalent) and 8% of methacrylic acid copolymer type B (Eudragit™ S100 or equivalent), all percentages expressed based on the total weight of the final modified release particles of sodium oxybate.

According to an embodiment, the modified release particles of gamma-hydroxybutyrate consist of 10.5% w/w of microcrystalline cellulose spheres with a volume mean diameter of about 95 microns to about 170 microns, layered with 56.5% w/w of sodium oxybate mixed with 3% w/w of Povidone™ K30 and finally coated with a coating composition consisting of 18% w/w of hydrogenated vegetable oil (Lubritab™ or equivalent), 4% of methacrylic acid copolymer type C (Eudragit™ L100-55 or equivalent) and 8% of methacrylic acid copolymer type B (Eudragit™ S100 or equivalent), all percentages expressed based on the total weight of the final modified release particles of sodium oxybate.

According to another embodiment, the modified release particles of gamma-hydroxybutyrate consist of 11.3% w/w of microcrystalline cellulose spheres with a volume mean diameter of about 95 microns to about 450 microns, layered with 60.5% w/w of gamma-hydroxybutyrate mixed with 3.2% w/w of Povidone™ K30 and finally coated with a coating composition consisting of 15% w/w of hydrogenated vegetable oil (Lubritab™ or equivalent), 0.75% of methacrylic acid copolymer type C (Eudragit™ L100-55 or equivalent) and 9.25% of methacrylic acid copolymer type B (Eudragit™ S100 or equivalent).

According to another embodiment, the modified release particles of gamma-hydroxybutyrate consist of 11.3% w/w of microcrystalline cellulose spheres with a volume mean diameter of about 95 microns to about 170 microns, layered with 60.5% w/w of gamma-hydroxybutyrate mixed with 3.2% w/w of Povidone™ K30 and finally coated with a coating composition consisting of 15% w/w of hydrogenated vegetable oil (Lubritab™ or equivalent), 0.75% of methacrylic acid copolymer type C (Eudragit™ L100-55 or equivalent) and 9.25% of methacrylic acid copolymer type B (Eudragit™ S100 or equivalent).

According to another embodiment, the modified release particles of gamma-hydroxybutyrate consist of 11.3% w/w of microcrystalline cellulose spheres with a volume mean diameter of about 95 microns to about 450 microns, layered with 60.5% w/w of sodium oxybate mixed with 3.2% w/w of Povidone™ K30 and finally coated with a coating composition consisting of 15% w/w of hydrogenated vegetable oil (Lubritab™ or equivalent), 0.75% of methacrylic acid copolymer type C (Eudragit™ L100-55 or equivalent) and 9.25% of methacrylic acid copolymer type B (Eudragit™ S100 or equivalent).

According to another embodiment, the modified release particles of gamma-hydroxybutyrate consist of 11.3% w/w of microcrystalline cellulose spheres with a volume mean diameter of about 95 microns to about 170 microns, layered with 60.5% w/w of sodium oxybate mixed with 3.2% w/w of Povidone™ K30 and finally coated with a coating composition consisting of 15% w/w of hydrogenated vegetable oil (Lubritab™ or equivalent), 0.75% of methacrylic acid copolymer type C (Eudragit™ L100-55 or equivalent) and 9.25% of methacrylic acid copolymer type B (Eudragit™ S100 or equivalent).

Further provided herein is a modified release formulation of gamma-hydroxybutyrate comprising immediate release and modified release portions, wherein the immediate release portion comprises particles of gamma- hydroxybutyrate, and the modified release portion comprises particles of gamma-hydroxybutyrate coated with a coating comprising: a polymer carrying free carboxylic groups, and a hydrophobic compound having a melting point equal or greater than 40° C., wherein the Tₘₐₓ, Cₘₐₓ, or AUC_{inf} of the modified release formulation is bioequivalent to the modified release formulation when concomitantly administered with divalproex sodium.

### Packaging

The composition of gamma-hydroxybutyrate may be supplied in sachets or stick-packs comprising a particulate formulation. The sachets may be available in several different doses, comprising gamma-hydroxybutyrate in amounts equivalents to 0.5 g, 1.0 g, 1.5 g, 3.0 g, 4.5 g, 6.0 g, 7.5 g, 9.0 g, 10.5 g and/or 12 g of sodium oxybate. Depending on the dose required, one or more of these sachets may be opened, and its contents mixed with tap water to provide the nightly dose of gamma-hydroxybutyrate.

### Methods of Treatment

Provided herein are methods for treating a human patient suffering from one or more symptoms of narcolepsy by orally administering a single daily dose to the human patient a full dosage amount of a pharmaceutical composition comprising gamma-hydroxybutyrate concomitantly with divalproex sodium. In some embodiments, the method may be effective to treat a disorder including but not limited to narcolepsy in a human patient in need thereof. Treatment of narcolepsy may include improvement (e.g., reduction) in one or more symptoms such as cataplexy, excessive daytime sleepiness, disrupted nighttime sleep, hypnagogic hallucinations, or sleep paralysis. In some examples, the human patient may be a human subject. Further provided herein is a method of treating a disorder treatable with gamma-hydroxybutyrate in a human subject in need thereof comprising orally administering a single daily dose to the human amounts of gamma-hydroxybutyrate equivalent to from 3.0 to 12.0 g of sodium oxybate in the composition concomitantly with divalproex sodium. Further provided herein are methods of treating narcolepsy, types 1 and/or 2, by orally administering a therapeutically effective amount of a gamma-hydroxybutyrate formulation characterized by the novel gamma-hydroxybutyrate pharmacokinetic properties of the composition when co-administered with divalproex sodium without reducing the dosage of gamma-hydroxybutyrate that would be administered absent the divalproex sodium. In an embodiment, the composition of the present invention is effective to treat narcolepsy Type 1 or Type 2, wherein the treatment of narcolepsy is defined as reducing excessive daytime sleepiness, reducing the frequency of cataplectic attacks, reducing disrupted nighttime sleep, reducing hypnagogic hallucinations, or reducing sleep paralysis. The therapeutically effective amount may include equivalents from 3.0 to 12.0 g of sodium oxybate. In various embodiments, the therapeutically effective amount is 4.5, 6.0, 7.5 or 9.0 g of sodium oxybate. In one embodiment, the therapeutically effective amount is 6 g or 9 g of sodium oxybate. In various embodiments, the formulation includes sodium oxybate present in a unit dose of at least 4.5 g, at least 6.0 g, at least 7.5 g, or at least 9.0 g. The effectiveness of the treatment may be measured by one or any combination of the following criteria:
Increase the mean sleep latency, as determined on the Maintenance of Wakefulness Test (MWT).
Improve the Clinical Global Impression (CGI) rating of sleepiness.
Decrease the number of cataplexy attacks (NCA) determined from the cataplexy frequency item in the Sleep and Symptoms Daily Diary.
Decrease the disturbed nocturnal sleep (DNS), the disturbed nocturnal events or the adverse respiratory events as determined by polysomnographic (PSG) measures of sleep fragmentation
Decrease the excessive daytime sleepiness (EDS) as measured by patient report via the Epworth Sleepiness Scale (ESS).
Decrease the daytime sleepiness as measured by the Maintenance of Wakefulness Test based on EEG measures of wakefulness.
Decrease PSG transitions from N/2 to N/3 and REM sleep to wake and N1 sleep (as determined by C Iber, S Ancoli-Israel, A Chesson, SF Quan. The AASM Manual for the Scoring of Sleep and Associated Events. Westchester, IL: American Academy of Sleep Medicine; 2007).
Decrease the number of arousals or wakenings, obtained from a PSG as defined by the American Academy of Sleep Medicine
Improve the sleep quality, obtained from one or more of (i) the Sleep and Symptom Daily Diary, (ii) Visual Analog Scale (VAS) for sleep quality and sleep diary, and (iii) VAS for the refreshing nature of sleep
Decrease the Hypnagogic Hallucinations (HH) or sleep paralysis (SP) symptoms in NT1 narcolepsy patients, as measured by the Sleep and Symptom Daily Diary

In an embodiment, the treatment using the composition co-administered with divalproex sodium is superior, as measured by any one or combination of the foregoing criteria, to an equal dose of the composition administered without divalproex sodium.

In some examples, the method includes treatment of narcolepsy Type 1 or Type 2 wherein, compared to a dosing regimen consisting of reducing the dosage sodium oxybate when concomitantly administering with divalproex sodium, a single daily dose administration of a therapeutically effective amount of the formulation of the invention concomitantly administered with divalproex sodium has been shown to not require a reduction in the sodium oxybate dosage.

Further provided herein are methods of treating narcolepsy, cataplexy, or excessive daytime sleepiness in a human subject including concomitantly administering to the subject a once-nightly dose of gamma-hydroxybutyrate and a dose of divalproex sodium, wherein the concomitant administration results in comparable systemic exposure to gamma-hydroxybutyrate as shown by plasma Cₘₐₓ and AUC values, as compared to administering gamma-hydroxybutyrate alone. The bioavailability of the once-nightly dose of gamma-hydroxybutyrate may not be affected by concomitantly administering with the dose of divalproex sodium. Concomitantly administering the gamma-hydroxybutyrate with divalproex sodium may not affect the pharmacokinetics of divalproex sodium as compared to administering divalproex sodium alone. In some embodiments, there is no drug-drug interaction between the once-nightly dose of gamma-hydroxybutyrate and the divalproex sodium.

In some embodiments, no dose adjustment may be made to the once-nightly dose of gamma-hydroxybutyrate or the dose of divalproex sodium for the concomitant administration.

In some embodiments, the concomitant administration may result in in no impairment of attention or working memory to the human subject.

The pharmacokinetic profile of the gamma-hdyroxybutyrate concomitantly administered with divalproex sodium may have the following characteristics.

In some embodiments, the Tₘₐₓ of the once-nightly gamma-hydroxybutyrate may be about 2.0 hours. In some embodiments, concomitantly administering the gamma-hydroxybutyrate with divalproex sodium may result in a Tₘₐₓ for divalproex sodium that is bioequivalent to the Tₘₐₓ when administering divalproex sodium alone.

In some embodiments, the Cₘₐₓ of the once-nightly dose of gamma-hdyroxybutyrate may be about 59 to about 97 μg/mL. Thus, in some examples, the Cₘₐₓ of the once-nightly dose of gamma-hydroxybutyrate may be 59 μg/mL, 60 μg/mL, 61 μg/mL, 62 μg/mL, 63 μg/mL, 64 μg/mL, 65 μg/mL, 66 μg/mL, 67 μg/mL, 68 μg/mL, 69μ g/mL, 70μ g/mL, 71μ g/mL, 72μ g/mL, 73μ g/mL, 74μ g/mL, 75μ g/mL, 76μ g/mL, 77 μg/mL, 78 μg/mL, 79 μg/mL, 80 μg/mL, 81 μg/mL, 82 μg/mL, 83 μg/mL, 84 μg/mL, 85 μg/mL, 86 μg/mL, 87 μg/mL, 88 μg/mL, 89 μg/mL, 90 μg/mL, 91 μg/mL, 92 μg/mL, 93 μg/mL, 94 μg/mL, 95 μg/mL, 96 μg/mL, or 97 μg/mL. In an exemplary embodiment, the Cₘₐₓ of the once-nightly dose of gamma-hdyroxybutyrate is about 78 μg/mL±19.

In some embodiments, the AUCo last of the once-nightly dose of gamma-hydroxybutyrate may be about 220 μg/mL·h to about 512 μg/mL·h. Thus, in some examples, the AUC₀₋ₗₐₛₜ of the once-nightly dose of gamma-hydroxybutyrate may be about 220 μg/mL·h, 250 μg/mL·h, 275 μg/mL·h, 300 μg/mL·h, 325 μg/mL·h, 350 μg/mL·h, 375 μg/mL·h, 400 μg/mL·h, 425 μg/mL·h, 450 μg/mL·h, 475 μg/mL·h, 500 μg/mL·h, or 512 μg/mL·h. In an exemplary embodiment, the AUC₀₋ₗₐₛₜ of the once-nightly dose of gamma-hydroxybutyrate is 366 μg/mL·h±146.

In some embodiments, the AUCo inf of the once-nightly dose of gamma-hydroxybutyrate may be about 220 μg/mL·h to about 512 μg/mL·h. Thus, in some examples, the AUCo-inf of the once-nightly dose of gamma-hydroxybutyrate may be about 220 μg/mL·h, 250 μg/mL·h, 275 μg/mL·h, 300 μg/mL·h, 325 μg/mL·h, 350 μg/mL·h, 375 μg/mL·h, 400 μg/mL·h, 425 μg/mL·h, 450 μg/mL·h, 475 μg/mL·h, 500 μg/mL·h, or 512 μg/mL·h. In an exemplary embodiment, the AUCo-inf of the once-nightly dose of gamma-hydroxybutyrate is 366 μg/mL·h +146.

In some embodiments, the AUCo-8 of the once-nightly dose of gamma-hydroxybutyrate may be about 222 μg/mL·h to about 488 μg/mL·h. Thus, in some examples, the AUCo-8 of the once-nightly dose of gamma-hydroxybutyrate may be about 222 μg/mL·h, 250 μg/mL·h, 275 μg/mL·h, 300 μg/mL·h, 325 μg/mL·h, 35 0μg/mL·h, 375 μg/mL·h, 400 μg/mL·h, 425 μg/mL·h, 450 μg/mL·h, 475 μg/mL·h, or 488 μg/mL·h. In an exmpalry embodiment, the AUCo-8 of the once-nightly dose of gamma-hydroxybutyrate is 355 μg/mL·h±133.

In some emboidments, the Geometric least sqares (LS) mean Cₘₐₓ (μg/mL) of the once-nightly dose of gamma-hydroxybutyrate may be about 75.62. IN some aspects, the Point Estimate (PE) providing the geometric mean ratio of Cₘₐₓ of the once-nightly dose of gamma-hdyroxybutyrate when concomitantly administered divided by the Cₘₐₓ of the once-nightly dose of gamma-hydroxybutyrate when administered alone may be about 98.46.

Further provided herein is a method for treating a patient suffering from one or more symptoms of narcolepsy, the method comprising: orally administering to the patient a full dosage amount of a pharmaceutical composition comprising gamma-hydroxybutyrate (GHB); and concomitantly administering a dosage of divalproex sodium (DVP), wherein the dosage of the GHB composition results in a Tₘₐₓ, Cₘₐₓ, or AUC_{inf} bioequivalent to the same dosage of the gamma-hydroxybutyrate composition administered alone. The dosage of the GHB composition may be present in a unit dose of of at least 4.5 g, at least 6.0 g, at least 7.5 g, or at least 9.0 g.

In some emboidments, the dosage of the gamma-hydroxybuytrate composition may result in a Tₘₐₓ bioequivalent ot the Tₘₐₓ as depicted in FIG. 2A. In some embodiments, the dosage of the gamma-hydroxybuytrate composition may result in a Cₘₐₓ bioequivalent ot the Cₘₐₓ as depicted in FIG. 2B.

In some emboidments, the dosage of the gamma-hydroxybutyrate composition may result in a Cₘₐₓ decrease of approximately 5% as compared to the same dosage of the gamma-hdyroxybutyrate composition administered alone.

In some emboidments, the dosage of the gamma-hydroxybutyrate composition may result in a AUC_{inf} bioequivalent to the AUC_{inf} as depicted in FIG. 2C.

### EXAMPLES

### Example 1. Formulations

Tables 1a-1d provide the qualitative and quantitative compositions of sodium oxybate IR microparticles, MR microparticles, and mixtures of IR and MR microparticles. The physical structure of the microparticles showing the qualitative and quantitative composition of the IR and MR microparticles is depicted in FIG. 1.

Briefly, sodium oxybate immediate release (IR) microparticles were prepared as follows: 1615.0 g of sodium oxybate and 85.0 g of polyvinylpyrrolidone (Povidone K30-Plasdone™ K29/32 from ISP) were solubilized in 1894.3 g of absolute ethyl alcohol and 1262.9 g of water. The solution was entirely sprayed onto 300 g of microcrystalline cellulose spheres (Cellets™ 127) in a fluid bed spray coater apparatus. IR Microparticles with volume mean diameter of about 270 microns were obtained.

Sodium oxybate modified release (MR) microparticles were prepared as follows: 22.8 g of methacrylic acid copolymer Type C (Eudragit™ L100-55), 45.8 g of methacrylic acid copolymer Type B (Eudragit™ S100), 102.9 g of hydrogenated cottonseed oil (Lubritab™), were dissolved in 1542.9 g of isopropanol at 78° C. The solution was sprayed entirely onto 400.0 g of the sodium oxybate IR microparticles described above in a fluid bed spray coater apparatus with an inlet temperature of 48° C., spraying rate around 11 g per min and atomization pressure of 1.3 bar. MR microparticles were dried for two hours with inlet temperature set to 56° C. MR microparticles with mean volume diameter of about 320 microns were obtained.

The finished composition, which contains a 50:50 mixture of MR and IR microparticles calculated on their sodium oxybate content, was prepared as follows: 353.36 g of the above IR microparticles, 504.80 g of the above MR microparticles, 14.27 g of malic acid (D/L malic acid), 6.34 g of xanthan gum (Xantural™ 75 from Kelco), 9.51 g of carrageenan gum (Viscarin™ PH209 from FMC Biopolymer), 9.51 g of hydroxyethylcellulose (Natrosol™ 250M from Ashland) and 4.51 g of magnesium stearate were mixed. Individual samples of 7.11 g (corresponding to a 4.5 g dose of sodium oxybate with half of the dose as immediate-release fraction and half of the dose as modified release fraction) were weighed.

TABLE 1a
Composition of IR Microparticles

| Component | Function | Quantity per 2.25 g dose (g) |
|---|---|---|
| Sodium oxybate | Drug substance | 2.25 |
| Microcrystalline | Core | 0.418 |
| cellulose spheres | | |
| Povidone K30 | Binder and excipient | 0.118 |
| | in diffusion coating | |
| Ethyl alcohol | Solvent | Eliminated during processing |
| Purified water | Solvent | Eliminated during processing |
| Total | | 2.786 |

TABLE 1b
Composition of MR Microparticles

| | | Quantity per |
|---|---|---|
| Component | Function | 4.5 g dose (g) |
| IR Microparticles | Core of MR | 2.786 |
| | microparticles | |
| Hydrogenated Vegetable Oil | Coating excipient | 0.716 |
| Methacrylic acid Copolymer | Coating excipient | 0.159 |
| Type C | | |
| Methacrylic acid Copolymer | Coating excipient | 0.318 |
| Type B | | |
| Isopropyl alcohol | Solvent | Eliminated during |
| | | processing |
| Total | | 3.981 |

TABLE 1c
Quatitative Finished Composition

| | | Quantity per |
|---|---|---|
| Component | Function | 4.5 g dose (g) |
| MR microparticles | Modified release fraction | 3.981 |
| | of sodium oxybate | |
| IR microparticles | Immediate release | 2.786 |
| | fraction of sodium | |
| | oxybate | |
| Malic acid | Acidifying agent | 0.113 |
| Xanthan gum | Suspending agent | 0.050 |
| Hydroxyethylcellulose | Suspending agent | 0.075 |
| Carrageenan gum | Suspending agent | 0.075 |
| Magnesium stearate | Lubricant | 0.036 |
| Total | | 7.116 |

TABLE 1d
Quantitative finished composition

| | | Quantity per |
|---|---|---|
| Component | Function | 4.5 g dose (g) |
| Sodium oxybate | Drug substance | 4.5 |
| Microcrystalline cellulose spheres | Core | 0.836 |
| Povidone K30 | Binder | 0.237 |
| Hydrogenated Vegetable Oil | Coating excipient | 0.716 |
| Methacrylic acid Copolymer Type C | Coating excipient | 0.159 |
| Methacrylic acid Copolymer Type B | Coating excipient | 0.318 |
| Malic acid | Acidifying agent | 0.113 |
| Xanthan gum | Suspending agent | 0.050 |
| Hydroxyethylcellulose | Suspending agent | 0.075 |
| Carrageenan gum | Suspending agent | 0.075 |
| Magnesium stearate | Lubricant | 0.036 |
| Total | | 7.116 |

### Example 1bis. Alternative Formulation

An alternative formulation to the formulation described in Example 1 is described in Example 1bis.

Sodium oxybate immediate release (IR) microparticles were prepared by coating the IR microparticles described in Example 1 with a top coat layer. Microparticles were prepared as follows: 170.0 of hydroxypropyl cellulose (Klucel™ EF Pharm from Hercules) were solubilized in 4080.0 g of acetone. The solution was entirely sprayed onto 1530.0 g of the IR microparticles of Example 1 in a fluid bed spray coater apparatus. IR Microparticles with volume mean diameter of about 298 microns were obtained (see Table 1bis-a).

Sodium oxybate modified release (MR) microparticles were prepared as described in example 1 (see Table 1b).

The finished composition, which contains a 50:50 mixture of MR and IR microparticles based on their sodium oxybate content, was prepared as follows: 412.22 g of the above IR microparticles, 530.00 g of the above MR microparticles, 29.96 g of malic acid (D/L malic acid), 4.96 g of xanthan gum (Xantural™ 75 from Kelco), 4.96 g of colloidal silicon dioxide (Aerosil™ 200 from Degussa) and 9.92 g of magnesium stearate were mixed. Individual samples of 7.45 g (corresponding to a 4.5 g dose of sodium oxybate with half of the dose in an immediate-release fraction and half of the dose in a modified release fraction) were weighed (see Table 1bis-b and 1bis-c).

TABLE 1bis-a
Composition of IR Microparticles

| | | Quantity per |
|---|---|---|
| Component | Function | 2.25 g dose (g) |
| Sodium oxybate | Drug substance | 2.25 |
| Microcrystalline cellulose | Core | 0.418 |
| spheres | | |
| Povidone K30 | Binder and excipient in | 0.118 |
| | diffusion coating | |
| Hydroxypropyl cellulose | Top coat | 0.310 |
| Ethyl alcohol | Solvent | Eliminated during |
| | | processing |
| Purified water | Solvent | Eliminated during |
| | | processing |
| Acetone | Solvent | Eliminated during |
| | | processing |
| Total | | 3.096 |

TABLE 1bis-b
Qualitative Finished Composition

| | | Quantity per |
|---|---|---|
| Component | Function | 4.5 g dose (g) |
| MR microparticles | Modified release fraction | 3.981 |
| | of sodium oxybate | |
| IR microparticles | Immediate release fraction | 3.096 |
| | of sodium oxybate | |
| Malic acid | Acidifying agent | 0.225 |
| Xanthan gum | Suspending agent | 0.037 |
| Colloidal silicon dioxide | Gliding agent | 0.037 |
| Magnesium stearate | Lubricant | 0.075 |
| Total | | 7.451 |

TABLE 1bis-c
Quantitative finished composition

| | | Quantity per |
|---|---|---|
| Component | Function | 4.5 g dose (g) |
| Sodium oxybate | Drug substance | 4.5 |
| Microcrystalline cellulose spheres | Core | 0.836 |
| Povidone K30 | Binder | 0.237 |
| Hydroxypropyl cellulose | Top coat | 0.310 |
| Hydrogenated Vegetable Oil | Coating excipient | 0.716 |
| Methacrylic acid Copolymer Type C | Coating excipient | 0.159 |
| Methacrylic acid Copolymer Type B | Coating excipient | 0.318 |
| Malic acid | Acidifying agent | 0.225 |
| Xanthan gum | Suspending agent | 0.037 |
| Colloidal silicon dioxide | Gliding agent | 0.037 |
| Magnesium stearate | Lubricant | 0.075 |
| Total | | 7.451 |

Compared to the finished composition described in Example 1, this alternative composition has the following characteristics: same MR microparticles, same IR microparticles but with a top coat, increased amount of malic acid, only one suspending agent (xanthan gum) and presence of a glidant.

### Example 2. In Vivo Pharmacokinetic Study of FT218 with and without DVP

Pharmacokinetic testing was undertaken in vivo in healthy human volunteers for a test product with the finished composition of Example 1 (FT218) co-administered with DVP. The study was designed to describe the magnitude of PK changes in FT218 when co-administrated with divalproex sodium ER evening dose. A total of 24 healthy male subjects between 18 and 55 years of age and with a BMI between 19.1 and 28.0 kg/m² participated in the study. One subject withdrew consent on Day 9 (pre-co-administration) and therefore, n=23 were administered FT218 with a 1250 mg/day divalproex sodium ER and n=24 were administered FT218 without DVP.

The study included a sequential, three period design with a single-dose administration of 6 g FT218 on Day 1 (Period 1), once daily 1250 mg divalproex sodium ER administration from Day 2-11 (Period 2), and FT218 and divalproex sodium ER co-administration on Day 12 (Period 3). All administrations were performed in the evening, 2 hours after the completion of dinner. Overall, no major safety issues were observed during this study and no SAEs or AESIs occurred.

Following administration of 6 g FT218 in the evening of Day 1, quantifiable concentrations of GHB were observed after 10 minutes (the first sampling point) for all subjects. Concentrations of GHB increased with maximum geometric mean concentration of 71.2 μg/mL reached at approximately 1 hour after administration. After reaching the peak concentration, GHB concentrations gradually decreased. Plasma concentrations of GHB were quantifiable in all subjects until at least 8 hours postdose.

The concentration versus time curves of FT218 with and without DVP are presented in FIGS. 1A and 1B. The derived PK parameters are summarized below (Table 2).

Co-administration of a single dose of 6 g FT218 with divalproex sodium ER in the evening increased AUCo-t and AUCo-inf for GHB by approximately 17%. The 90% CIs of the ratio of the mean of Cₘₐₓ and AUC were contained within the standard bioequivalence range (80.00%-125.00%), respecting the bioequivalence criteria. Cₘₐₓ was not affected by the co-administration of divalproex sodium ER. Tₘₐₓ were comparable with or without co-administration of divalproex sodium ER.

TABLE 2
Mean PK Parameters

| | | Cmax | AUC₀₋ₗₐₛₜ | AUC_{0-inf} | AUC₀₋₈ₕ | C8h |
|---|---|---|---|---|---|---|
| | Tmax (h) | (μg/mL) ± SD | (μg/mL · h) ± SD | (μg/mL · h) ± SD | (μg/mL · h) ± SD | (μg/mL) ± SD |
| Treatment | [min-max] | (CV) | (CV) | (CV) | (CV) | (CV) |
| FT218 | 1.3 | 80 ± 20 | 307 ± 107 | 308 ± 107 | 304 ± 105 | 4.0 ± 4.3 |
| alone | [0.3-3.0] | (25%) | (35%) | (35%) | (34%) | (108%) |
| n = 24 | | | | | | |
| FT 218 + | 2.0 | 78 ± 19 | 366 ± 146 | 366 ± 146 | 355 ± 133 | 9.8 ± 10.7 |
| DVP | [0.3-3.5] | (25%) | (40%) | (40%) | (38%) | (108) |
| n = 23 | | | | | | |

### Example 3. Comparison of FT218 with and without DVP

To compare the effect of DVP on FT218, the mean values for Tₘₐₓ, Cₘₐₓ, and AUC_{inf} with FT218 alone and FT218 with DVP were plotted together. The effect of DVP on FT218 is shown in FIGS. 2A, 2B, and 2C. FIG. 2A shows the mean Tₘₐₓ values for each patient when administered FT218 alone and when co-administered with DVP. FIG. 2B shows the mean Cₘₐₓ values for each patient when administered FT218 alone and when co-administered with DVP. FIG. 2C shows the mean AUC_{inf} values for each patient when administered FT218 alone and when co-administered with DVP. In comparison, FT218 with DVP appears to demonstrate similar behavior as FT218 alone. Thus, FT218 with and without DVP appear to have similar PK profiles.

The Point Estimate (PE) providing the geometric mean ratio of FT218+DVP/FT218 (alone) and 90% confidence intervals (CI) of the PE are shown below (Table 3). The 90% CIs of the ratio of the mean of Cₘₐₓ and AUC were contained within the standard bioequivalence range (80.00%-125.00%), respecting the bioequivalence criteria. The 90% CI for the T/R ratio did not include 100 for AUCo int (T/R ratio [90% CI]: 116.74 [111.03-122.73]) and AUCo-t (T/R ratio [90% CI]: 116.67[111.18-122.44]), indicating an increase of AUC by approximately 17%. Cₘₐₓ for both treatments was similar (T/R ratio [90% CI]: 98.46 [91.58-105.85]). Thus, Cₘₐₓ, AUC₀₋ₗₐₛₜ and AUCo-int 90% confidence intervals are within the 80-125% bioequivalence range. The tmax for GHB was comparable for both treatments. This was confirmed by non-parametric statistical analysis.

TABLE 3
PK Analysis

| PK | PE (ratio geomean) | 90% CI | 90% CI |
|---|---|---|---|
| Parameter | (FT218 + DVP/FT218 alone) | Lower | Upper |
| Cₘₐₓ | 98.46 | 91.58 | 105.85 |
| AUC₀₋ₗₐₛₜ | 116.67 | 111.18 | 122.44 |
| AUC_{0-inf} | 116.52 | 111.07 | 122.23 |

### Example 4. Comparison of DVP with and without FT218

Following administration of 1250 mg divalproex sodium ER in the evening of Day 11, the geometric mean concentration of valproic acid increased from 58.5 μg/mL at baseline to a maximum geometric mean concentration of 79.5 μg/mL 14 hours after administration. After reaching the peak concentration, geometric mean concentrations of valproic acid returned to 57.9 μg/mL at 24 hours after administration.

Following administration of 1250 mg divalproex sodium ER in the evening of Day 12, in the presence of concentrations of GHB, the geometric mean concentration of valproic acid increased from 57.9 μg/mL at baseline to a maximum geometric mean concentration of 77.0 μg/mL 14 hours after administration. After reaching the peak concentration, geometric mean concentrations of valproic acid returned to 64.0 μg/mL at 24 hours after administration.

For subjects who received both divalproex sodium ER treatments on Day 11 (without FT218) and Day 12 (with FT218), respectively, and who were included in the statistical analysis (N=23), the geometric means of Cₘₐₓ and AUC₀₋₂₄ for valproic acid on both days were compared. The 90% CIs of the ratio of the mean of Cₘₐₓ and AUC₀₋₂₄ were contained within the standard bioequivalence range (80.00%-125.00%), respecting the bioequivalence criteria. AUC₀₋₂₄ for both treatments was similar (T/R ratio [90% CI]: 97.28 [94.59-100.04]). For Cₘₐₓ, the 90% CI for the T/R ratio did not include 100 (T/R ratio [90% CI]: 94.82 [91.03-98.76]), indicating a decrease of Cₘₐₓ by approximately 5%. The tmax for valproic acid was comparable for both treatments. This was confirmed by non-parametric statistical analysis.

To compare the effect of FT218 on DVP, the concentration versus time curves for a 1250 mg dose of DVP administered alone (Day 11) and co-administered with FT218 (Day 12) were plotted together.

FIG. 3A shows the mean PK profiles of DVP with and without co-administration with FT218. FIG. 3B shows individual PK profiles of DVP with and without co-administration with FT218. This appears to demonstrate a similar DVP profile with or without FT218.

### Example 5. Comparisons with DDI Study of Xyrem®

To compare the effect of DVP on FT218 and Xyrem®, the geometric LS mean AUC_{inf} values for FT218 with and without DVP were compared with the geometric LS mean AUC_{inf} values for Xyrem® with and without DVP from a drug-drug interaction (DDI) study for Xyrem (Eller et al, 2013). Tables 4 and 5 below provide the comparison. Table 4 shows that the Cₘₐₓ and AUC_{inf} for 6 g FT218 with 1250 mg/day DVP are within the 80%-125% bioequivalence range of the Cₘₐₓ and AUC_{inf} for a 6 g dose of FT218 alone, while Table 5 shows the AUC_{inf} for two 3 g doses Xyrem® with 1250 mg/day DVP is above the bioequivalence range for AUC_{inf}. Specifically, Xyrem administered with DVP without adjusting dosage resulted in about 127% AUC_{inf} of Xyrem alone while FT218 administered with DVP without adjusting dosage resulted in about 117% AUC_{inf} of FT218 alone. Thus, Xyrem with DVP is outside bioequivalence limits, while FT218 with DVP is within the bioequivalence limits.

TABLE 4
DDI study, PKFT218-1901 (evening dosing)

| | Geometric | Geometric |
|---|---|---|
| | LS mean AUC_{0-inf} | LS mean Cₘₐₓ |
| Treatment | (μg/mL · h) | (μg/mL) |
| FT218 alone (6 g) | 290.48 | 76.81 |
| n = 23 | | |
| FT218 (6 g) + DVP | 338.46 | 75.62 |
| (1250 mg/day) | | |
| n = 23 | | |
| PE (FT218 DVP/ | 116.52 | 98.46 |
| FT218 alone) (%) | | |

TABLE 5
DDI study Xyrem ®, Eller et al. 2013

| | Geometric LS mean | Geometric |
|---|---|---|
| | AUC_{0-inf} | LS mean Cₘₐₓ |
| Treatment | (μg/mL · h) | (μg/mL) |
| Xylem ® alone | 275.6 | Not detailed |
| (twice 3 g) | | |
| n = 20 | | |
| Xylem ® (twice | 349.7 | Not detailed |
| 3 g) + DVP (1250 | | |
| mg/day) | | |
| n = 20 | | |
| PE (Xyreme ® + | 126.9 | Not detailed |
| DVP/Xyrem ® | | |
| alone) (%) | | |

### Example 6. In Vivo Pharmacokinetic Study of FT218 with and without DVP Administered in the Morning

Pharmacokinetic testing was undertaken in vivo in healthy human volunteers for a test product with the finished composition of Example 1 (FT218) co-administered with DVP. The study was an open-label, sequential study to assess the drug-drug interaction of divalproex sodium extended release (ER) at steady-state on the FT218 formulation administered at a single 6 g morning dose in healthy volunteers. A total of 22 healthy subjects participated in the study. A total of 22 subjects completed the study as per protocol and 21 subjects were evaluable for the GHB PK statistical analysis. The FT218 was administered in the morning, 2h post-morning meal, with or without 1250 mg/day divalproex sodium ER.

The study included a sequential, three period design with a single-dose administration of 6 g FT218 on Day 1 (Period 1), once daily 1250 mg divalproex sodium ER administration from Day 2-11 (Period 2), and FT218 and divalproex sodium ER co-administration on Day 12 (Period 3). All administrations were performed in the morning, 2 hours after the completion of a morning meal. Overall, no major safety issues were observed during this study and no SAEs or AESIs occurred.

Following administration of 6 g FT218 in the morning of Day 1, quantifiable concentrations of GHB were observed after 10 minutes (the first sampling point) for all subjects. After reaching the peak concentration, GHB concentrations gradually decreased. Plasma concentrations of GHB were quantifiable in all subjects until at least 8 hours postdose.

The concentration versus time curves of FT218 with and without DVP are presented in FIGS. 4A and 4B. The derived PK parameters are summarized below (Table 6).

The 90% CIs of the ratio of the mean of Cₘₐₓ and AUC were contained within the standard bioequivalence range (80.00%-125.00%), respecting the bioequivalence criteria. Cₘₐₓ was not affected by the co-administration of divalproex sodium ER. Tₘₐₓ was comparable with or without co-administration of divalproex sodium ER.

TABLE 6
Mean PK Parameters

| | | Cmax | AUC₀₋ₗₐₛₜ | AUC_{0-inf} | AUC₀₋₈ₕ | C8h |
|---|---|---|---|---|---|---|
| | Tmax (h) | (μg/mL) ± SD | (μg/mL · h) ± SD | (μg/mL · h) ± SD | (μg/mL · h) ± SD | (μg/mL) ± SD |
| Treatment | [min-max] | (CV) | (CV) | (CV) | (CV) | (CV) |
| FT218 | 0.76 | 107 ± 25 | 333 ± 113 | 334 ± 113 | 332 ± 112 | 1.70 ± 1.70 |
| alone | [0.3-3.03] | (24%) | (34%) | (34%) | (34%) | (100%) |
| n = 22 | | | | | | |
| FT 218 + | 1.0 | 108 ± 19 | 403 ± 140 | 404 ± 140 | 399 ± 134 | 5.62 ± 7.23 |
| DVP | [0.33-4.5] | (18%) | (35%) | (35%) | (34%) | (24%) |
| n = 22 | | | | | | |

### Example 7. Comparison of FT218 with and without DVP

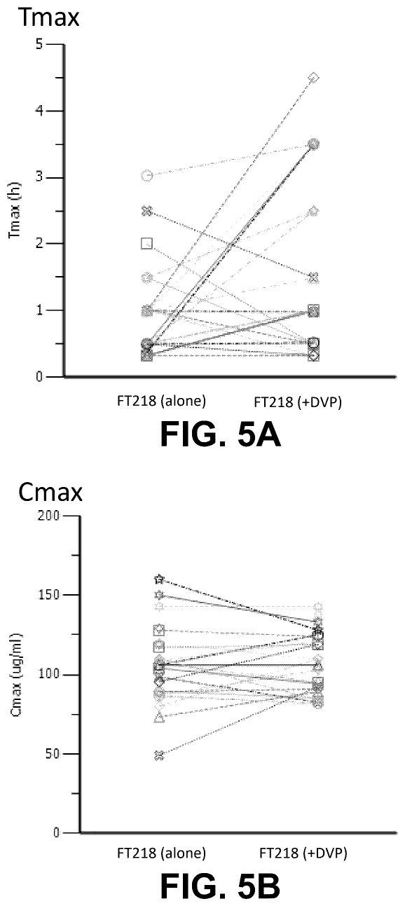

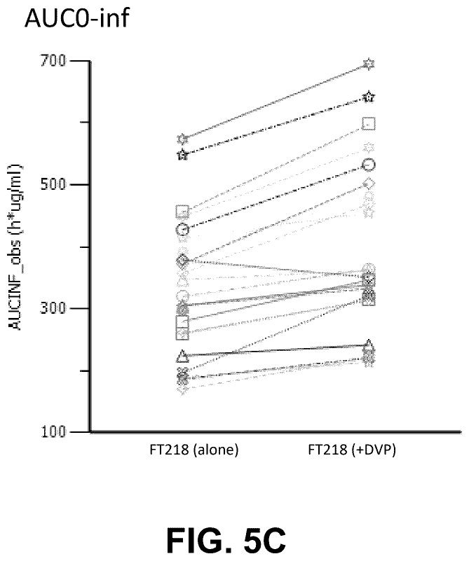

To compare the effect of DVP on FT218 administered once in the morning, the mean values for Tₘₐₓ, Cₘₐₓ, and AUC_{inf} with FT218 alone and FT218 with DVP were plotted together. The effect of DVP on FT218 is shown in FIGS. 5A, 5B, and 5C. FIG. 5A shows the mean Tₘₐₓ values for each patient when administered FT218 alone and when co-administered with DVP. FIG. 5B shows the mean Cₘₐₓ values for each patient when administered FT218 alone and when co-administered with DVP. FIG. 5C shows the mean AUC_{inf} values for each patient when administered FT218 alone and when co-administered with DVP. In comparison, FT218 with DVP appears to demonstrate similar behavior as FT218 alone. Thus, FT218 with and without DVP appear to have similar PK profiles when administered in the morning.

The Point Estimate (PE) providing the geometric mean ratio of FT218+DVP/FT218 (alone) and 90% confidence intervals (CI) of the PE are shown below (Table 7). The 90% CIs of the ratio of the mean of Cₘₐₓ and AUC were contained within the standard bioequivalence range (80.00%-125.00%), respecting the bioequivalence criteria. The results indicate an increase of AUC by approximately 18%. Cₘₐₓ for both treatments was similar. Thus, Cₘₐₓ, AUC₀₋ₗₐₛₜ and AUC_{0-inf} 90% confidence intervals are within the 80-125% bioequivalence range.

TABLE 7
PK Analysis

| PK | PE (ratio geomean) | 90% CI | 90% CI |
|---|---|---|---|
| Parameter | (FT218 + DVP/FT218 alone) | Lower | Upper |
| Cₘₐₓ | 103.93 | 96.11 | 112.49 |
| AUC₀₋ₗₐₛₜ | 118.82 | 113.94 | 123.91 |
| AUC_{0-inf} | 118.76 | 113.88 | 123.84 |

### Example 8. Comparison of DVP with and without FT218

Following administration of 1250 mg divalproex sodium ER in the morning of Day 11, the geometric mean concentration of valproic acid increased to a maximum geometric mean concentration of 72.43 μg/mL.

For subjects who received both divalproex sodium ER treatments on Day 11 (without FT218) and Day 12 (with FT218), respectively, and who were included in the statistical analysis (N=22), the geometric means of Cₘₐₓ and AUC₀₋₂₄ for valproic acid on both days were compared. The 90% CIs of the ratio of the mean of Cₘₐₓ and AUC₀₋₂₄ were contained within the standard bioequivalence range (80.00%-125.00%), respecting the bioequivalence criteria.

To compare the effect of FT218 on DVP, the concentration versus time curves for a 1250 mg dose of DVP administered alone (Day 11) and co-administered with FT218 (Day 12) were plotted together.

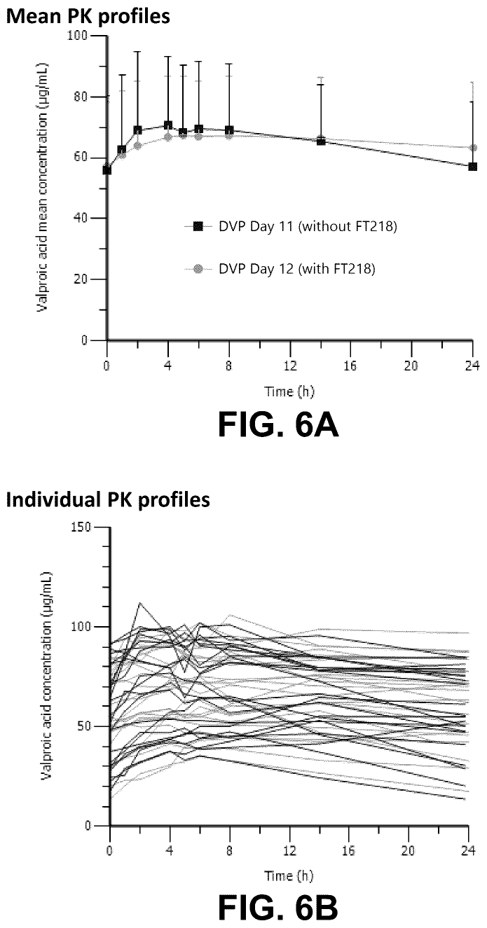

FIG. 6A shows the mean PK profiles of DVP with and without co-administration with FT218 in the morning. FIG. 6B shows individual PK profiles of DVP with and without co-administration with FT218 in the morning. This appears to demonstrate a similar DVP profile with or without FT218.

### Example 9. Inter-Study Comparison of FT218 Alone and with DVP

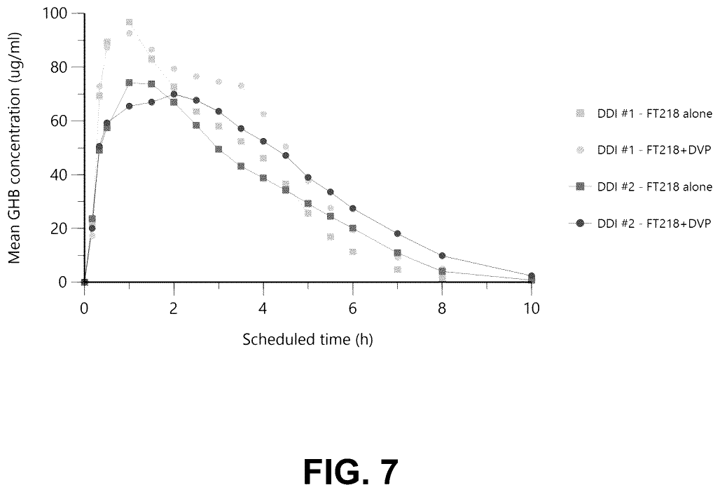

FIG. 7 shows a mean concentration versus time curve for FT218 administered alone and with DVP in two separate studies (DDI #1, DDI #2).

DDI #1 was an open-label, sequential study to assess the drug-drug interaction of divalproex sodium extended release (ER) at steady-state on the FT218 formulation administered at a single 6 g morning dose in healthy volunteers. In DDI #1, the FT218 was administered in the morning, 2h post-morning meal, with or without 1250 mg/day divalproex sodium ER. Examples 6-8 show the results from DDI #1.

DDI #2 was an open-label, sequential study to assess the drug-drug interaction of divalproex sodium extended release (ER) at steady-state on the FT218 formulation administered at a single 6 g evening dose in healthy male volunteers. In DDI #2, the FT218 was administered in the evening, 2h post-evening meal, with or without 1250 mg/day divalproex sodium ER. Examples 2-4 show the results from DDI #2.

As seen in FIG. 7, the comparison of DDI #1 and DDI #2 shows that the interaction between FT218 and DVP has a similar effect on the GHB concentration, independent of time of administration. Therefore, FT218 may be co-administered once daily (morning or evening) with DVP without having to reduce the FT218 dosage.

Throughout this application, various publications are referenced. The disclosures of these publications in their entireties are hereby incorporated by reference into this application in order to more fully describe the state of the art to which this invention pertains. It will be apparent to those skilled in the art that various modifications and variations may be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### Example 10. Labelling for FT218 Embodiment

### Warning: Central Nervous System (CNS) Depression and Abuse and Misuse

### Central Nervous System Depression

FT218 (sodium oxybate) is a CNS depressant. In clinical trials at recommended doses, obtundation and clinically significant respiratory depression occurred in adult patients treated with immediate-release sodium oxybate [see Warnings and Precautions (5.1)]. Many patients who received sodium oxybate during clinical trials in narcolepsy were receiving central nervous system stimulants.

### Abuse and Misuse

FT218 (sodium oxybate) is the sodium salt of gamma-hydroxybutyrate (GHB). Abuse or misuse of illicit GHB, either alone or in combination with other CNS depressants, is associated with CNS adverse reactions, including seizure, respiratory depression, decreases in the level of consciousness, coma, and death.

Because of the risks of CNS depression and abuse and misuse, FT218 is available only through a restricted program under a Risk Evaluation and Mitigation Strategy (REMS) called the FT218 REMS.

### Indications and Usage

FT218 is indicated for the treatment of cataplexy or excessive daytime sleepiness (EDS) in adults with narcolepsy.

### Dosage and Administration

### Dosing Information

The recommended starting dosage is 4.5 grams (g) once per night administered orally. Increase the dosage by 1.5 g per night at weekly intervals to the effective dosage range of 6 g to 9 g once per night orally. The dosage may be gradually titrated based on efficacy and tolerability. Doses higher than 9 g per night have not been studied and should not ordinarily be administered.

### Important Administration Instructions

FT218 is taken orally as a single dose at bedtime. Prepare the dose of FT218 prior to bedtime. Prior to ingestion, the dose of FT218 should be suspended in approximately 1/3 cup (approximately 80 mL) of water in the mixing cup provided. Do not use hot water. After mixing, consume FT218 within 30 minutes.

Take FT218 at least 2 hours after eating.

Patients should take FT218 while in bed and lie down immediately after dosing as FT218 may cause them to fall asleep abruptly without first feeling drowsy. Patients will often fall asleep within 5 minutes of taking FT218, and will usually fall asleep within 15 minutes, though the time it takes any individual patient to fall asleep may vary from night to night. Rarely, patients may take up to 2 hours to fall asleep. Patients should remain in bed following ingestion of FT218.

Switching Patients from Immediate-Release Sodium Oxybate

Patients who are currently being treated with immediate-release sodium oxybate may be switched to FT218 at the nearest equivalent dosage in g per night (e.g., 7.5 g sodium oxybate divided into two 3.75 g doses per night to 7.5 g FT218 once per night).

### Dosage Forms and Strengths

For extended-release oral suspension: FT218 is a white to off-white

powder provided in packets of 4.5 g, 6 g, 7.5 g, or 9 g of sodium oxybate.

### CONTRAINDICATIONS

FT218 is contraindicated for use in:
combination with sedative hypnotics combination with alcohol. patients with succinic semialdehyde dehydrogenase deficiency

### Warnings and Precautions

### Central Nervous System Depression

FT218 is a central nervous system (CNS) depressant. In adult clinical trials at recommended doses, obtundation and clinically significant respiratory depression occurred in patients treated with immediate-release sodium oxybate. FT218 is contraindicated in combination with alcohol and sedative hypnotics. The concurrent use of FT218 with other CNS depressants, including but not limited to opioid analgesics, benzodiazepines, sedating antidepressants or antipsychotics, sedating anti-epileptic drugs, general anesthetics, muscle relaxants, and/or illicit CNS depressants, may increase the risk of respiratory depression, hypotension, profound sedation, syncope, and death. If use of these CNS depressants in combination with FT218 is required, dose reduction or discontinuation of one or more CNS depressants (including FT218) should be considered. No pharmacokinetic interaction has been observed between FT218 and divalproex sodium, and no dose adjustment is recommended for their concomitant use based on this pharmacokinetic observation. In addition, if short-term use of an opioid (e.g., post-or perioperative) is required, interruption of treatment with FT218 should be considered. Consumption of alcohol while taking FT218 may also result in a more rapid release of the dose of sodium oxybate.

Healthcare providers should caution patients about operating hazardous machinery, including automobiles or airplanes, until they are reasonably certain that FT218 does not affect them adversely (e.g., impair judgment, thinking, or motor skills). Patients should not engage in hazardous occupations or activities requiring complete mental alertness or motor coordination, such as operating machinery or a motor vehicle or flying an airplane, for at least 6 hours after taking FT218. Patients should be queried about CNS depression-related events upon initiation of FT218 therapy and periodically thereafter.

FT218 is available only through a restricted program under a REMS.

### Abuse and Misuse

FT218 is a Schedule III controlled substance. The active ingredient of FT218, sodium oxybate, is a form of gamma-hydroxybutyrate (GHB), a Schedule I controlled substance. Abuse of illicit GHB, either alone or in combination with other CNS depressants, is associated with CNS adverse reactions, including seizure, respiratory depression, decreases in the level of consciousness, coma, and death. The rapid onset of sedation, coupled with the amnestic features of GHB, particularly when combined with alcohol, has proven to be dangerous for the voluntary and involuntary user (e.g., assault victim). Because illicit use and abuse of GHB have been reported, physicians should carefully evaluate patients for a history of drug abuse and follow such patients closely, observing them for signs of misuse or abuse of GHB (e.g., increase in size or frequency of dosing, drug-seeking behavior, feigned cataplexy).

FT218 is available only through a restricted program under a REMS.

### FT218 REMS

FT218 is available only through a restricted distribution program

called the FT218 REMS because of the risks of central nervous system depression and abuse and misuse.

Notable requirements of the FT218 REMS include the following:
Healthcare providers who prescribe FT218 are specially certified.
FT218 will be dispensed only by pharmacies that are specially certified.
FT218 will be dispensed and shipped only to patients who are enrolled in the FT218 REMS with documentation of safe use conditions.

Further information is available at www.FT218 REMS.com or by calling 1-877-453-1029.

### Respiratory Depression and Sleep-Disordered Breathing

FT218 may impair respiratory drive, especially in patients with compromised respiratory function. In overdoses of oxybate with illicit use of GHB, life-threatening respiratory depression has been reported.

Increased apnea and reduced oxygenation may occur with FT218 administration. A significant increase in the number of central apneas and clinically significant oxygen desaturation may occur in patients with obstructive sleep apnea treated with FT218.

In an adult study assessing the respiratory-depressant effects of immediate-release sodium oxybate at doses up to 9 g per night in 21 patients with narcolepsy, no dose-related changes in oxygen saturation were demonstrated in the group as a whole. One of four patients with preexisting moderate-to-severe sleep apnea had significant worsening of the apnea/hypopnea index during treatment.

In an adult study assessing the effects of immediate-release sodium oxybate 9 g per night in 50 patients with obstructive sleep apnea, immediate-release sodium oxybate did not increase the severity of sleep-disordered breathing and did not adversely affect the average duration and severity of oxygen desaturation overall. However, there was a significant increase in the number of central apneas in patients taking immediate-release sodium oxybate, and clinically significant oxygen desaturation (≤55%) was measured in three patients (6%) after administration, with one patient withdrawing from the study, and two continuing after single brief instances of desaturation.

In adult clinical trials in 128 patients with narcolepsy administered immediate-release sodium oxybate, two subjects had profound CNS depression, which resolved after supportive respiratory intervention. Two other patients discontinued immediate-release sodium oxybate because of severe difficulty breathing and an increase in obstructive sleep apnea. In two controlled trials assessing polysomnographic (PSG) measures in adult patients with narcolepsy administered immediate-release sodium oxybate, 40 of 477 patients were included with a baseline apnea/hypopnea index of 16 to 67 events per hour, indicative of mild to severe sleep-disordered breathing. None of the 40 patients had a clinically significant worsening of respiratory function, as measured by apnea/hypopnea index and pulse oximetry at doses of 4.5 g to 9 g per night. In adult clinical trials of FT218 in patients with narcolepsy, no subjects with apnea/hypopnea indexes greater than 15 were allowed to enroll.

Prescribers should be aware that sleep-related breathing disorders tend to be more prevalent in obese patients, in men, in postmenopausal women not on hormone replacement therapy, and among patients with narcolepsy.

### Depression and Suicidality

Depression, and suicidal ideation and behavior, can occur in patients treated with FT218.

In an adult clinical trial in patients with narcolepsy (n=212) administered FT218, there were no suicide attempts, but one patient developed suicidal ideation at the 9 g dose. In adult clinical trials in patients with narcolepsy (n=781) administered immediate-release sodium oxybate, there were two suicides and two attempted suicides in patients treated with immediate-release sodium oxybate, including three patients with a previous history of depressive psychiatric disorder. Of the two suicides, one patient used immediate-release sodium oxybate in conjunction with other drugs. Immediate-release sodium oxybate was not involved in the second suicide. Adverse reactions of depression were reported by 7% of 781 patients treated with immediate-release sodium oxybate, with four patients (<1%) discontinuing because of depression. In most cases, no change in immediate-release sodium oxybate treatment was required.

In a controlled trial in adults with narcolepsy administered FT218 (n=212) where patients were titrated from 4.5 g to 9 g per night, the incidences of depression were 0% at 4.5 g, 1% at 6 g, 1.1% at 7.5 g, and 1.3% at 9 g. In a controlled adult trial, with patients randomized to fixed doses of 3 g, 6 g, or 9 g per night immediate-release sodium oxybate or placebo, there was a single event of depression at the 3 g per night dose. In another adult controlled trial, with patients titrated from an initial 4.5 g per night starting dose of immediate-release sodium oxybate, the incidences of depression were 1.7%, 1.5%, 3.2%, and 3.6% for the placebo, 4.5 g, 6 g, and 9 g per night doses, respectively.

The emergence of depression in patients treated with FT218 requires careful and immediate evaluation. Patients with a previous history of a depressive illness and/or suicide attempt should be monitored carefully for the emergence of depressive symptoms while taking FT218.

### Other Behavioral or Psychiatric Adverse Reactions

Other behavioral and psychiatric adverse reactions can occur in patients taking FT218.

During adult clinical trials in patients with narcolepsy administered FT218, 2% of 107 patients treated with FT218 experienced a confusional state. During adult clinical trials in patients with narcolepsy administered immediate-release sodium oxybate, 3% of 781 patients treated with immediate-release sodium oxybate experienced confusion, with incidence generally increasing with dose.

No patients treated with FT218 discontinued treatment because of confusion. Less than 1% of patients discontinued the immediate-release sodium oxybate because of confusion. Confusion was reported at all recommended doses of immediate-release sodium oxybate from 6 g to 9 g per night. In a controlled trial in adults where patients were randomized to immediate-release sodium oxybate in fixed total daily doses of 3 g, 6 g, or 9 g per night or placebo, a dose-response relationship for confusion was demonstrated, with 17% of patients at 9 g per night experiencing confusion. In that controlled trial, the confusion resolved in all cases soon after termination of treatment. In one trial where immediate-release sodium oxybate was titrated from an initial 4.5 g per night dose, there was a single event of confusion in one patient at the 9 g per night dose. In the majority of cases in all adult clinical trials in patients with narcolepsy administered immediate-release sodium oxybate, confusion resolved either soon after termination of dosing or with continued treatment.

Anxiety occurred in 7.5% of 107 patients treated with FT218 in the adult trial in patients with narcolepsy. Anxiety occurred in 5.8% of the 874 patients receiving immediate-release sodium oxybate in adult clinical trials in another population.

Other psychiatric reactions reported in adult clinical trials in patients with narcolepsy administered FT218 included irritability, emotional disorder, panic attack, agitation, delirium, and obsessive thoughts. Other neuropsychiatric reactions reported in adult clinical trials in patients with narcolepsy administered immediate-release sodium oxybate and in the post-marketing setting for immediate-release sodium oxybate include hallucinations, paranoia, psychosis, aggression, and agitation.

The emergence or increase in the occurrence of behavioral or psychiatric events in patients taking FT218 should be carefully monitored.

### Parasomnias

Parasomnias can occur in patients taking FT218.

Sleepwalking, defined as confused behavior occurring at night and at times associated with wandering, was reported in 3% of 107 patients with narcolepsy treated with FT218. No patients treated with FT218 discontinued due to sleepwalking.

Sleepwalking was reported in 6% of 781 patients with narcolepsy treated with immediate-release sodium oxybate in adult controlled and long-term open-label studies, with <1% of patients discontinuing due to sleepwalking. In controlled trials, rates of sleepwalking were similar for patients taking placebo and patients taking immediate-release sodium oxybate. It is unclear if some or all of the reported sleepwalking episodes correspond to true somnambulism, which is a parasomnia occurring during non-REM sleep, or to any other specific medical disorder. Five instances of sleepwalking with potential injury or significant injury were reported during a clinical trial of immediate-release sodium oxybate in patients with narcolepsy.

Parasomnias, including sleepwalking, have also been reported in the postmarketing experience with immediate-release sodium oxybate. Therefore, episodes of sleepwalking should be fully evaluated, and appropriate interventions considered.

### Use in Patients Sensitive to High Sodium Intake

FT218 has a high salt content. In patients sensitive to salt intake

(e.g., those with heart failure, hypertension, or renal impairment), consider the amount of daily sodium intake in each dose of FT218. Table 10a provides the approximate sodium content per FT218 dose.

TABLE 10a
Approximate Sodium Content per Total Nightly Dose
of FT218 (g = grams)

| FT218 Dose | Sodium Content/Total Nightly Exposure |
|---|---|
| 4.5 g per night | 820 mg |
| 6 g per night | 1100 mg |
| 7.5 g per night | 1400 mg |
| 9 g per night | 1640 mg |

### Adverse Reactions

The following clinically significant adverse reactions appear in other sections of the labeling:
CNS Depression
Abuse and Misuse
Respiratory Depression and Sleep-Disordered Breathing
Depression and Suicidality
Other Behavioral or Psychiatric Adverse Reactions
Parasomnias
Use in Patients Sensitive to High Sodium Intake

### Clinical Trials Experience

Because clinical trials are conducted under widely varying conditions, adverse reaction rates observed in the clinical trials of a drug cannot be directly compared to rates in the clinical trials of another drug and may not reflect the rates observed in clinical practice.

FT218 was studied in one placebo-controlled trial (Study 1) [see Clinical Studies (14)] in 212 patients with narcolepsy (107 patients treated with FT218 and 105 with placebo).

### Adverse Reactions Leading to Treatment Discontinuation

Of the 212 patients with narcolepsy treated with FT218, 15.9%

discontinued because of adverse reactions, compared to 1.9% of patients receiving placebo. The most common adverse reaction leading to discontinuation was xx (x %). For FT218, 5.6% of patients discontinued due to adverse reactions on 4.5 g, 4.1% on 6 g, 4.5% on 7.5 g, and 3.9% on 9 g dose.

### Most Common Adverse Reactions

The most common adverse reactions (incidence >5% and greater than placebo) reported for any dose of FT218 were nausea, dizziness, enuresis, headache, and vomiting.

### Adverse Reactions Occurring at an Incidence of 2% or Greater

Table 10b lists adverse reactions occurring in 2% or more of

FT218 -treated patients on any individual dose and at a rate greater than placebo-treated patients in Study 1.

TABLE 10b

| | | FT218 | FT218 | FT218 | FT218 |
|---|---|---|---|---|---|
| | Placebo | 4.5 g | 6 g | 7.5 g | 9 g |
| Adverse | (N = | (N = | (N = | (N = | (N = |
| Reaction | 105) % | 107) % | 97) % | 88) % | 77) % |

Gastrointestinal disorders

| | | | | | |
|---|---|---|---|---|---|
| Vomiting | 2 | 3 | 3 | 6 | 5 |
| Nausea | 3 | 6 | 8 | 7 | 1 |

Investigations

| | | | | | |
|---|---|---|---|---|---|
| Weight | 0 | 1 | 0 | 0 | 4 |
| Decreased | | | | | |

Metabolism and Nutritional Disorders

| | | | | | |
|---|---|---|---|---|---|
| Decreased | 0 | 4 | 4 | 3 | 3 |
| appetite | | | | | |

Nervous System Disorders

| | | | | | |
|---|---|---|---|---|---|
| Dizziness | 0 | 6 | 4 | 6 | 5 |
| Somnolence | 1 | 0 | 1 | 2 | 4 |
| Headache | 6 | 7 | 5 | 6 | 0 |

Psychiatric Disorders

| | | | | | |
|---|---|---|---|---|---|
| Enuresis | 0 | 2 | 4 | 9 | 9 |
| Anxiety | 1 | 3 | 1 | 3 | 1 |
| Somnambulism | 0 | 1 | 2 | 0 | 0 |

### Dose-Response Information

In clinical trials in adult patients with narcolepsy, a dose-response relationship was observed for enuresis and somnolence.

### Additional Adverse Reactions

Adverse reactions observed in clinical studies with immediate-release sodium oxybate (≥2%), but not observed in Study 1 at a frequency of higher than 2%, and which may be relevant for FT218: diarrhea, abdominal pain upper, dry mouth, pain, feeling drunk, peripheral edema, cataplexy, muscle spasms, pain in extremity, tremor, disturbance in attention, paresthesia, sleep paralysis, disorientation, irritability, and hyperhidrosis.

### Postmarketing Experience

The following adverse reactions have been identified during postapproval use of sodium oxybate. Because these reactions are reported voluntarily from a population of uncertain size, it is not always possible to reliably estimate their frequency or establish a causal relationship to drug exposure:

Arthralgia, decreased appetite, fall*, fluid retention, hangover, headache, hypersensitivity, hypertension, memory impairment, nocturia, panic attack, vision blurred, and weight decreased.

*The sudden onset of sleep in patients taking sodium oxybate, including in a standing position or while rising from bed, has led to falls complicated by injuries, in some cases requiring hospitalization.

### Drug Interactions

Alcohol, Sedative Hypnotics, and CNS Depressants

FT218 is contraindicated for use in combination with alcohol or

sedative hypnotics. Use of other CNS depressants may potentiate the CNS-depressant effects of FT218. Consumption of alcohol while taking FT218 may also result in a more rapid release of the dose of sodium oxybate.

### Use in Specific Populations

### Pregnancy

### Risk Summary

There are no adequate data on the developmental risk associated

with the use of sodium oxybate in pregnant women. Oral administration of sodium oxybate to pregnant rats (150, 350, or 1,000 mg/kg/day) or rabbits (300, 600, or 1,200 mg/kg/day) throughout organogenesis produced no clear evidence of developmental toxicity; however, oral administration to rats throughout pregnancy and lactation resulted in increased stillbirths and decreased offspring postnatal viability and growth, at a clinically relevant dose.

In the U.S. general population, the estimated background risk of major birth defects and miscarriage in clinically recognized pregnancies is 2-4% and 15-20%, respectively. The background risk of major birth defects and miscarriage for the indicated population is unknown.

### Clinical Considerations

### Labor or Delivery

FT218 has not been studied in labor or delivery. In obstetric p anesthesia using an injectable formulation of sodium oxybate, newborns had stable cardiovascular and respiratory measures but were very sleepy, causing a slight decrease in Apgar scores. There was a fall in the rate of uterine contractions 20 minutes after injection. Placental transfer is rapid and gamma-hydroxybutyrate (GHB) has been detected in newborns at delivery after intravenous administration of GHB to mothers. Subsequent effects of sodium oxybate on later growth, development, and maturation in humans are unknown.

### Data

### Animal Data

Oral administration of sodium oxybate to pregnant rats (150, 350, or 1,000 mg/kg/day) or rabbits (300, 600, or 1,200 mg/kg/day) throughout organogenesis produced no clear evidence of developmental toxicity. The highest doses tested in rats and rabbits were approximately 1 and 3 times, respectively, the maximum recommended human dose (MRHD) of 9 g per night on a body surface area (mg/m²) basis.

Oral administration of sodium oxybate (150, 350, or 1,000 mg/kg/day) to rats throughout pregnancy and lactation resulted in increased stillbirths and decreased offspring postnatal viability and body weight gain at the highest dose tested. The no-effect dose for pre-and postnatal developmental toxicity in rats is less than the MRHD on a mg/m² basis.

### Lactation

### Risk Summary

GHB is excreted in human milk after oral administration of sodium oxybate. There is insufficient information on the risk to a breastfed infant, and there is insufficient information on milk production in nursing mothers. The developmental and health benefits of breastfeeding should be considered along with the mother's clinical need for FT218 and any potential adverse effects on the breastfed infant from FT218 or from the underlying maternal condition.

### Pediatric Use

Safety and effectiveness of FT218 in pediatric patients have not been established.

### Juvenile Animal Toxicity Data

In a study in which sodium oxybate (0, 100, 300, or 900 mg/kg/day) was orally administered to rats during the juvenile period of development (postnatal days 21 through 90), mortality was observed at the two highest doses tested. Deaths occurred during the first week of dosing and were associated with clinical signs (including decreased activity and respiratory rate) consistent with the pharmacological effects of the drug. Reduced body weight gain in males and females and delayed sexual maturation in males were observed at the highest dose tested.

### Geriatric Use

Clinical studies of FT218 or immediate-release sodium oxybate in patients with narcolepsy did not include sufficient numbers of subjects age 65 years and older to determine whether they respond differently from younger subjects. In controlled trials of immediate-release sodium oxybate in another population, 39 (5%) of 874 patients were 65 years or older. Discontinuations of treatment due to adverse reactions were increased in the elderly compared to younger adults (21% vs. 19%). Frequency of headaches was markedly increased in the elderly (39% vs. 19%). The most common adverse reactions were similar in both age categories. In general, dose selection for an elderly patient should be cautious, usually starting at the low end of the dosing range, reflecting the greater frequency of decreased hepatic, renal, or cardiac function, and of concomitant disease or other drug therapy.

### Hepatic Impairment

Because of an increase in exposure to FT218, FT218 should not be initiated in patients with hepatic impairment because appropriate dosage adjustments for initiation of FT218 cannot be made with the available dosage strengths. Patients with hepatic impairment who have been titrated to a maintenance dosage of another oxybate product can be switched to FT218 if the appropriate dosage strength is available.

### Drug Abuse and Dependence

### Controlled Substance

FT218 is a Schedule III controlled substance under the Federal Controlled Substances Act. Non-medical use of FT218 could lead to penalties assessed under the higher Schedule I controls.

### Abuse

FT218 (sodium oxybate), the sodium salt of GHB, produces dose-dependent central nervous system effects, including hypnotic and positive subjective reinforcing effects. The onset of effect is rapid, enhancing its potential for abuse or misuse.

Drug abuse is the intentional non-therapeutic use of a drug product or substance, even once, for its desirable psychological or physiological effects. Misuse is the intentional use, for therapeutic purposes of a drug by an individual in a way other than prescribed by a healthcare provider or for whom it was not prescribed. Drug misuse and abuse may occur with or without progression to addiction. Drug addiction is a cluster of behavioral, cognitive, and physiological phenomena that may include a strong desire to take the drug, difficulties in controlling drug use (e.g., continuing drug use despite harmful consequences, giving a higher priority to drug use than other activities and obligations), and possible tolerance or physical dependence.

The rapid onset of sedation, coupled with the amnestic features of GHB, particularly when combined with alcohol, has proven to be dangerous for the voluntary and involuntary user (e.g., assault victim).

Illicit GHB is abused in social settings primarily by young adults. Some of the doses estimated to be abused are in a similar dosage range to that used for treatment of patients with cataplexy. GHB has some commonalities with ethanol over a limited dose range, and some cross tolerance with ethanol has been reported as well. Cases of severe dependence and craving for GHB have been reported when the drug is taken around the clock. Patterns of abuse indicative of dependence include: 1) the use of increasingly large doses, 2) increased frequency of use, and 3) continued use despite adverse consequences.

Because illicit use and abuse of GHB have been reported, physicians should carefully evaluate patients for a history of drug abuse and follow such patients closely, observing them for signs of misuse or abuse of GHB (e.g., increase in size or frequency of dosing, drug-seeking behavior, feigned cataplexy). Dispose of FT218 according to state and federal regulations. It is safe to dispose of FT218 down the sanitary sewer.

### Dependence

### Dependence

Physical dependence is a state that develops as a result of physiological adaptation in response to repeated drug use, manifested by withdrawal signs and symptoms after abrupt discontinuation or a significant dose reduction of a drug. There have been case reports of withdrawal, ranging from mild to severe, following discontinuation of illicit use of GHB at frequent repeated doses (18 g to 250 g per day) in excess of the recommended dosage range. Signs and symptoms of GHB withdrawal following abrupt discontinuation included insomnia, restlessness, anxiety, psychosis, lethargy, nausea, tremor, sweating, muscle cramps, tachycardia, headache, dizziness, rebound fatigue and sleepiness, confusion, and, particularly in the case of severe withdrawal, visual hallucinations, agitation, and delirium. These symptoms generally abated in 3 to 14 days. In cases of severe withdrawal, hospitalization may be required. The discontinuation effects of FT218 have not been systematically evaluated in controlled clinical trials. In the clinical trial experience with immediate-release sodium oxybate in narcolepsy/cataplexy patients at recommended doses, two patients reported anxiety and one reported insomnia following abrupt discontinuation at the termination of the clinical trial; in the two patients with anxiety, the frequency of cataplexy had increased markedly at the same time.

### Tolerance

Tolerance is a physiological state characterized by a reduced response to a drug after repeated administration (i.e., a higher dose of a drug is required to produce the same effect that was once obtained at a lower dose). Tolerance to FT218 has not been systematically studied in controlled clinical trials. There have been some case reports of symptoms of tolerance developing after illicit use at dosages far in excess of the recommended FT218 dosage regimen. Clinical studies of immediate-release sodium oxybate in the treatment of alcohol withdrawal suggest a potential cross-tolerance with alcohol. The safety and effectiveness of FT218 in the treatment of alcohol withdrawal have not been established.

### Overdosage

### Human Experience

Information regarding overdose with FT218 is derived largely from reports in the medical literature that describe symptoms and signs in individuals who have ingested GHB illicitly. In these circumstances, the co-ingestion of other drugs and alcohol was common and may have influenced the presentation and severity of clinical manifestations of overdose.

In adult clinical trials of immediate-release sodium oxybate, two cases of overdose with sodium oxybate were reported. In the first case, an estimated dose of 150 g, more than 15 times the maximum recommended dose, caused a patient to be unresponsive with brief periods of apnea and to be incontinent of urine and feces. This individual recovered without sequelae. In the second case, death was reported following a multiple drug overdose consisting of sodium oxybate and numerous other drugs.

### Signs and Symptoms

Information about signs and symptoms associated with overdosage with FT218 derives from reports of illicit use of GHB. Patient presentation following overdose is influenced by the dose ingested, the time since ingestion, the co-ingestion of other drugs and alcohol, and the fed or fasted state. Patients have exhibited varying degrees of depressed consciousness that may fluctuate rapidly between a confusional, agitated combative state with ataxia and coma. Emesis (even when obtunded), diaphoresis, headache, and impaired psychomotor skills have been observed. No typical pupillary changes have been described to assist in diagnosis; pupillary reactivity to light is maintained. Blurred vision has been reported. An increasing depth of coma has been observed at higher doses. Myoclonus and tonic-clonic seizures have been reported.

Respiration may be unaffected or compromised in rate and depth. Cheyne-Stokes respiration and apnea have been observed. Bradycardia and hypothermia may accompany unconsciousness, as well as muscular hypotonia, but tendon reflexes remain intact.

### Recommended Treatment of Overdose

General symptomatic and supportive care should be instituted immediately, and gastric decontamination may be considered if co-ingestants are suspected. Because emesis may occur in the presence of obtundation, appropriate posture (left lateral recumbent position) and protection of the airway by intubation may be warranted. Although the gag reflex may be absent in deeply comatose patients, even unconscious patients may become combative to intubation, and rapid-sequence induction (without the use of sedative) should be considered. Vital signs and consciousness should be closely monitored. The bradycardia reported with GHB overdose has been responsive to atropine intravenous administration. No reversal of the central depressant effects of FT218 can be expected from naloxone or flumazenil administration. The use of hemodialysis and other forms of extracorporeal drug removal have not been studied in GHB overdose. However, due to the rapid metabolism of sodium oxybate, these measures are not warranted.

### Poison Control Center

As with the management of all cases of drug overdosage, the possibility of multiple drug ingestion should be considered. The healthcare provider is encouraged to collect urine and blood samples for routine toxicologic screening, and to consult with a regional poison control center (1-800-222-1222) for current treatment recommendations.

### Description

Sodium oxybate, a CNS depressant, is the active ingredient in FT218 for extended-release oral suspension. The chemical name for sodium oxybate is sodium 4-hydroxybutyrate. The molecular formula is C₄H₇NaO₃, and the molecular weight is 126.09 g/mole. The chemical structure is:

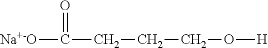

Sodium oxybate is a white to off-white solid powder.

Each packet of FT218 contains 4.5 g, 6 g, 7.5 g, or 9 g of sodium oxybate, equivalent to 3.7 g, 5.0 g, 6.2 g, or 7.4 g of oxybate, respectively. The inactive ingredients are carrageenan, hydrogenated vegetable oil, hydroxyethyl cellulose, magnesium stearate, malic acid, methacrylic acid copolymer, microcrystalline cellulose, povidone, and xanthan gum.

### Clinical Pharmacology

### Mechanism of Action

FT218 is a CNS depressant. The mechanism of action of FT218 in

the treatment of narcolepsy is unknown. Sodium oxybate is the sodium salt of gamma-hydroxybutyrate (GHB), an endogenous compound and metabolite of the neurotransmitter GABA. It is hypothesized that the therapeutic effects of FT218 on cataplexy and excessive daytime sleepiness are mediated through GABAB actions at noradrenergic and dopaminergic neurons, as well as at thalamo-cortical neurons.

### Pharmacokinetics

### Absorption

Following oral administration of FT218, the peak plasma

concentrations (Cₘₐₓ) following administration of one 6 g dose was 66 mcg/mL, and the time to peak plasma concentration (Tₘₐₓ) was 1.5 hours. Following oral administration of FT218, the plasma levels of GHB increased dose-proportionally for Cₘₐₓ, and more than dose-proportionally for AUC (respectively 2.0-fold and 2.3-fold increases as total daily dose is doubled from 4.5 g to 9 g).

### p Effect of Food

Administration of FT218 immediately after a high-fat meal resulted

in a mean reduction in Cₘₐₓ and AUC of GHB by 33% and 16%, respectively; average Tₘₐₓ increased from 0.5 hour to 1.5 hours.

### Effect of Ethanol

An in vitro study showed alcohol-induced dose-dumping of sodium oxybate from extended-release oral suspension at 1 hour in the presence of 40% alcohol, and approximately 60% increase of drug release at 2 hours in the presence of 20% alcohol.

### Effect of Water Temperature

An in vitro dissolution study showed that FT218 mixed with hot water (90° C.) resulted in a dose-dumping phenomenon for the release of sodium oxybate, whereas warm water (50° C.) did not significantly affect the drug release from the extended-release suspension.

### Distribution

GHB is a hydrophilic compound with an apparent volume of distribution averaging 190 mL/kg to 384 mL/kg. At GHB concentrations ranging from 3mcg/mL to 300 mcg/mL, less than 1% is bound to plasma proteins.

### Elimination

### Metabolism

Animal studies indicate that metabolism is the major elimination pathway for GHB, producing carbon dioxide and water via the tricarboxylic acid (Krebs) cycle, and secondarily by beta-oxidation. The primary pathway involves a cytosolic NADP+-linked enzyme, GHB dehydrogenase, which catalyzes the conversion of GHB to succinic semialdehyde, which is then biotransformed to succinic acid by the enzyme succinic semialdehyde dehydrogenase. Succinic acid enters the Krebs cycle where it is metabolized to carbon dioxide and water. A second mitochondrial oxidoreductase enzyme, a transhydrogenase, also catalyzes the conversion to succinic semialdehyde in the presence of a-ketoglutarate. An alternate pathway of biotransformation involves ß-oxidation via 3,4-dihydroxybutyrate to carbon dioxide and water. No active metabolites have been identified.

### Excretion

The clearance of GHB is almost entirely by biotransformation to carbon dioxide, which is then eliminated by expiration. On average, less than 5% of unchanged drug appears in human urine within 6 to 8 hours after dosing. Fecal excretion is negligible. GHB has an elimination half-life of 0.5 to 1 hour.

### Specific Population

### Geriatric Patients

There is limited experience with FT218 in the elderly. Results from a pharmacokinetic study of immediate-release sodium oxybate (n=20) in another studied population indicate that the pharmacokinetic characteristics of GHB are consistent among younger (age 48 to 64 years) and older (age 65 to 75 years) adults.

### Male and Female Patients

In a study of 18 female and 18 male healthy adult volunteers, no gender differences were detected in the pharmacokinetics of GHB following an immediate-release 4.5 g oral dose of sodium oxybate.

### Racial or Ethnic Groups

There are insufficient data to evaluate any pharmacokinetic differences among races.

Patients with Renal Impairment

No pharmacokinetic study in patients with renal impairment has been conducted.

Patients with Hepatic Impairment

The pharmacokinetics of GHB in 16 cirrhotic patients, half without ascites (Child's Class A) and half with ascites (Child's Class C), were compared to the kinetics in 8 subjects with normal hepatic function, after a single sodium oxybate oral dose of 25 mg/kg. AUC values were doubled in cirrhotic patients, with apparent oral clearance reduced from 9.1 mL/min/kg in healthy adults to 4.5 and 4.1 mL/min/kg in Class A and Class C patients, respectively. Elimination half-life was significantly longer in Class C and Class A patients than in control patients (mean t1/2 of 59 minutes and 32 minutes, respectively, versus 22 minutes in control patients). FT218 should not be initiated in patients with liver impairment.

### Drug Interaction Studies

In vitro studies with pooled human liver microsomes indicate that

sodium oxybate does not significantly inhibit the activities of the human isoenzymes CYP1A2, CYP2C9, CYP2C19, CYP2D6, CYP2E1, or CYP3A, up to the concentration of 3 mM (378 mcg/mL), a level considerably higher than levels achieved with the maximum recommended dose.

Drug interaction studies in healthy adults (age 18 to 50 years) were conducted with immediate-release sodium oxybate and diclofenac and ibuprofen:
Diclofenac: Co-administration of sodium oxybate (6 g per day as two equal doses of 3 grams dosed four hours apart) with diclofenac (50 mg/dose twice per day) showed no significant changes in systemic exposure to GHB. Co-administration did not appear to affect the pharmacokinetics of diclofenac.
Ibuprofen: Co-administration of sodium oxybate (6 g per day as two equal doses of 3 grams dosed four hours apart) with ibuprofen (800 mg/dose four times per day also dosed four hours apart) resulted in comparable systemic exposure to GHB, as shown by plasma Cₘₐₓ and AUC values. Co-administration did not affect the pharmacokinetics of ibuprofen.

Drug interaction studies in healthy adults demonstrated no pharmacokinetic interactions between immediate-release sodium oxybate and protriptyline hydrochloride, zolpidem tartrate, and modafinil. Also, there were no pharmacokinetic interactions with the alcohol dehydrogenase inhibitor fomepizole. However, pharmacodynamic interactions with these drugs cannot be ruled out. Alteration of gastric pH with omeprazole produced no significant change in the pharmacokinetics of GHB. In addition, drug interaction studies in healthy adults demonstrated no pharmacokinetic or clinically significant pharmacodynamic interactions between immediate-release sodium oxybate and duloxetine HCl.

### Nonclinical Toxicology

### Carcinogenesis, Mutagenesis, Impairment of Fertility

### Carcinogenesis

Administration of sodium oxybate to rats at oral doses of up to

1,000 mg/kg/day for 83 (males) or 104 (females) weeks resulted in no increase in tumors. Plasma exposure (AUC) at the highest dose tested was 2 times that in humans at the maximum recommended human dose (MRHD) of 9 g per night.

The results of 2-year carcinogenicity studies in mouse and rat with gamma-butyrolactone, a compound that is metabolized to sodium oxybate in vivo, showed no clear evidence of carcinogenic activity. The plasma AUCs of sodium oxybate achieved at the highest doses tested in these studies were less than that in humans at the MRHD.

### Mutagenesis

Sodium oxybate was negative in the in vitro bacterial gene mutation assay, an in vitro chromosomal aberration assay in mammalian cells, and in an in vivo rat micronucleus assay.

### Impairment of Fertility

Oral administration of sodium oxybate (150, 350, or 1,000 mg/kg/day) to male and female rats prior to and throughout mating and continuing in females through early gestation resulted in no adverse effects on fertility. The highest dose tested is approximately equal to the MRHD on a mg/m² basis.

### Clinical Studies

The effectiveness of FT218 for the treatment of cataplexy or excessive daytime sleepiness (EDS) in adults with narcolepsy has been established based on a double-blind, randomized, placebo-controlled, two-arm multi-center study to assess the efficacy and safety of a once-nightly administration of FT218 in patients with narcolepsy (Study 1; NCTXXXXXXXX).

A total of 212 patients were randomized to receive FT218 or placebo in a 1:1 ratio. The study was divided into four sequential study periods, and incorporated dose titration to stabilized dose administration of FT218 (4.5 g, 6 g, 7.5 g, and 9 g). There was a three-week screening period, a 13-week treatment period including up-titration over a period of eight weeks, five weeks of stable dosing at 9g/night, and a one-week follow-up period. Patients could be on concomitant stimulant as long as dosage was stable for 3 weeks prior to study start.

The three co-primary endpoints were the Maintenance of Wakefulness Test (MWT), Clinical Global Impression-Improvement (CGI-I), and mean change in weekly cataplexy attacks. The MWT measures latency to sleep onset (in minutes), averaged over five sessions at 2-hour intervals following nocturnal polysomnography. For each test session, patients were instructed to remain awake for as long as possible during 30-minute test sessions, and sleep latency was determined as the number of minutes patients could remain awake. The overall score was the mean sleep latency for the 5 sessions. The CGI-I was evaluated on a 7-point scale, centered at No Change, and ranging from Very Much Worse to Very Much Improved. Patients were rated by evaluators who based their assessments on the severity of narcolepsy at Baseline.

Demographic and mean baseline characteristics were similar for the FT218 and placebo groups. A total of 76% were narcolepsy type 1 (NT1) patients, and 24% were narcolepsy type 2 (NT2) patients. The mean age was 31 years, and 68% were female. Approximately 63% of patients were on concomitant stimulant use. The mean MWT at baseline was 5 minutes for the FT218 group, and 4.7 minutes for the placebo group. The mean number of cataplexy attacks per week at Baseline was 18.9in the FT218 group and 19.8 in the placebo group. A statistically significant improvement was seen on the MWT, CGI-I, and mean weekly cataplexy attacks, for the 6 g (Week 3), 7.5 g (Week 8), and 9 g (Week 13) dose of FT218, compared to the placebo group (see Table 10c, Table 10d, and Table 10e). Results (MWT and CGI-I) were consistent between NT1 and NT2 patients, as well as between patients on stimulants and those not on stimulants.

TABLE 10c

| | | Change from | Difference from | |
|---|---|---|---|---|
| | Treatment | Baseline | Placebo [95% | p-value |
| Dose | Group (N) | (Minutes) | CI] | |
| 6 g (Week 3) | FT218 (87) | 8.1 | 5.0 [2.90; 7.05] | <0.001 |
| | Placebo (88) | 3.1 | | |
| 7.5 g (Week | FT218 (76) | 9.6 | 6.2 [3.84; 8.58] | <0.001 |
| 8) | Placebo (78) | 3.3 | | |
| 9 g (Week | FT218 (68) | 10.8 | 6.1 [3.52, 8.75] | <0.001 |
| 13) | Placebo (78) | 4.7 | | |

Mean (SD) MWT at Baseline was 499 (3.15) minutes for the FT218 group and 4.73 (2.58) minutes for the placebo group

TABLE 10d

| | | Percentage of | | |
|---|---|---|---|---|
| | | Responders | | |
| | | (Much or | | |
| | Treatment | Very Much | Odds Ratio | |
| Dose | Group (N) | Improved) | [95% CI] | p-value |
| 6 g (Week 3) | FT218 (87) | 40 | 10.3 [3.93; 26.92] | <0.001 |
| | Placebo (87) | 6 | — | — |
| 7.5 g (Week | FT218 (75) | 64 | 5.7 [2.82; 11.40] | <0.001 |
| 8) | Placebo (81) | 22 | — | — |
| 9 g (Week | FT218 (69) | 73 | 5.6 [2.76; 11.23] | <0.001 |
| 13) | Placebo (79) | 32 | — | — |

TABLE 10e

| | Treatment | Change from | Difference from | |
|---|---|---|---|---|
| Dose | Group (N) | Baseline¹ | Placebo [95% CI] | p-value |
| 6 g (Week 3) | FT218 (73) | −7.4 | −4.8 [−7.03; | <0.001 |
| | | | −2.62] | |
| | Placebo (72) | −2.6 | — | — |
| 7.5 g (Week | FT218 (66) | −10.0 | −6.3 [−8.74; | <0.001 |
| 8) | | | −3.80] | |
| | Placebo (69) | −3.7 | — | — |
| 9 g (Week | FT218 (54) | −11.5 | −6.7 [−9.32; | <0.001 |
| 13) | | | −3.98] | |
| | Placebo (62) | −4.9 | — | — |

¹Mean (SD) number of cataplexy attacks per week at Baseline was 18.9 (8.7) in the FT218 group and 19.8 (8.9) in the placebo group.

### How Supplied/Storage and Handling

How Supplied FT218 is a blend of white to off-white granules for extended-release

oral suspension in water. Each carton contains either 7 or 30 packets of FT218, a mixing cup, Prescribing Information and Medication Guide, and Instructions for Use.

Dose packets contain a single dose of FT218 provided in 4.5 g, 6 g, 7.5 g, or 9 g doses.

| Strength | Package Size | NDC Number |
|---|---|---|
| 4.5 g | 7 packets | NDC 13551-001-07 |
| | 30 packets | NDC 13551-001-30 |
| 6 g | 7 packets | NDC 13551-002-07 |
| | 30 packets | NDC 13551-002-30 |
| 7.5 g | 7 packets | NDC 13551-003-07 |
| | 30 packets | NDC 13551-003-30 |
| 9 g | 7 packets | NDC 13551-004-07 |
| | 30 packets | NDC 13551-004-30 |

### Storage

Keep out of reach of children.

FT218 should be stored at 20° C. to 25° C. (68° F. to 77° F.); excursions

permitted to 15°° C. to 30° C. (59° F. to 86° F.) (see USP Controlled Room Temperature).

Suspensions should be consumed within 30 minutes.

### Handling and Disposal

FT218 is a Schedule III drug under the Controlled Substances Act.

FT218 should be handled according to state and federal regulations. It is safe to dispose of FT218 down the sanitary sewer.

### Patient Counseling Information

Advise the patient to read the FDA-approved patient labeling (Medication Guide and Instructions for Use).

### Central Nervous System Depression

Inform patients that FT218 can cause central nervous system depression, including respiratory depression, hypotension, profound sedation, syncope, and death. Instruct patients to not engage in activities requiring mental alertness or motor coordination, including operating hazardous machinery, for at least 6 hours after taking FT218. Instruct patients to inform their healthcare providers of all the medications they take.

### Abuse and Misuse

Inform patients that the active ingredient of FT218 is gamma-hydroxybutyrate (GHB), which is associated with serious adverse reactions with illicit use and abuse.

FT218 REMS

FT218 is available only through a restricted program called the FT218 REMS. Inform the patient of the following notable requirements:
FT218 is dispensed only by pharmacies that are specially certified
FT218 will be dispensed and shipped only to patients who are enrolled in the FT218 REMS

FT218 is available only from certified pharmacies participating in the program. Therefore, provide patients with the telephone number and website for information on how to obtain the product.

Alcohol or Sedative Hypnotics

Advise patients that alcohol and other sedative hypnotics should not be taken with FT218.

### Sedation

Inform patients that they are likely to fall asleep quickly after taking FT218 (often within 5 and usually within 15 minutes), but the time it takes to fall asleep can vary from night to night. The sudden onset of sleep, including in a standing position or while rising from bed, has led to falls complicated by injuries, in some cases requiring hospitalization. Instruct patients that they should remain in bed following ingestion of their dose.

### Food Effects on FT218

Inform patients that FT218 should be taken at least 2 hours after eating.

### Respiratory Depression and Sleep-Disordered Breathing

Inform patients that FT218 may impair respiratory drive, especially in patients with compromised respiratory function, and may cause apnea.

### Depression and Suicidality

Instruct patients to contact a healthcare provider immediately if they develop depressed mood, markedly diminished interest or pleasure in usual activities, significant change in weight and/or appetite, psychomotor agitation or retardation, increased fatigue, feelings of guilt or worthlessness, slowed thinking or impaired concentration, or suicidal ideation.

### Other Behavioral or Psychiatric Adverse Reactions

Inform patients that FT218 can cause behavioral or psychiatric adverse reactions, including confusion, anxiety, and psychosis. Instruct them to notify their healthcare provider if any of these types of symptoms occur.

### Sleepwalking

Instruct patients that FT218 has been associated with sleepwalking and other behaviors during sleep, and to contact their healthcare provider if this occurs.

### Sodium Intake

Instruct patients that FT218 contains a significant amount of sodium and patients who are sensitive to sodium intake (e.g., those with heart failure, hypertension, or renal impairment) should limit their sodium intake.

What is claimed is:

1. A method of decreasing excessive daytime sleepiness in a human subject, the method comprising:
administering to the subject a dose of gamma-hydroxybutyrate, wherein the administering occurs only once daily; and
co-administering a dose of divalproex sodium, wherein the co-administering results in comparable systemic exposure to gamma-hydroxybutyrate as shown by Cₘₐₓ, as compared to when the dose of gamma-hydroxybutyrate is administered alone.

2. The method of claim 1, wherein the co-administering achieves a mean Cₘₐₓ that is from 80% to 125% of the Cₘₐₓ achieved when the dose of gamma-hydroxybutyrate is administered without divalproex sodium.

3. The method of claim 1, wherein the co-administering achieves a mean AUC_{inf} that is from 80% to 125% of the AUC_{inf} achieved when the dose of gamma-hydroxybutyrate is administered without divalproex sodium.

4. The method of claim 1, wherein the co-administering achieves an AUC ratio range of 0.8 to 1.7 achieved when the dose of gamma-hydroxybutyrate is administered without divalproex sodium.

5. The method of claim 1 wherein the co-administering results in a Tₘₐₓ for divalproex sodium that is bioequivalent to the Tₘₐₓ of divalproex sodium when administered alone.

6. The method of claim 1, wherein the dose of gamma-hydroxybutyrate comprises an amount of gamma-hydroxybutyrate equivalent to 3.0 g, 4.5 g, 6.0 g, 7.5 g, 9.0 g, 10.5 g or 12 g of sodium oxybate.

7. The method of claim 1, wherein the administering occurs at bedtime.

8. The method of claim 1, wherein the gamma-hydroxybutyrate comprises the sodium salt of gamma-hydroxybutyric acid, the potassium salt of gamma-hydroxybutyric acid, the magnesium salt of gamma-hydroxybutyric acid, and the calcium salt of gamma-hydroxybutyric acid, and wherein the dose of gamma-hydroxybutyrate comprises an amount of the gamma-hydroxybutyrate equivalent to from 3 g to 6 g of sodium oxybate.

9. The method of claim 8, wherein the administering comprises administering a reduced dosage amount of the dose of gamma-hydroxybutyrate relative to a dosage amount of the dose of gamma-hydroxybutyrate that would be administered to the subject absent the co-administering.

10. The method of claim 9, wherein the dose of gamma-hydroxybutyrate is reduced by less than 5% relative to the dosage amount of the dose of gamma-hydroxybutyrate that would be administered to the subject absent the co-administering.

11. The method of claim 9, wherein the dose of gamma-hydroxybutyrate is reduced by at least 20% relative to the dosage amount of the dose of gamma-hydroxybutyrate that would be administered to the subject absent the co-administering.

12. The method of claim 1, wherein the administering comprises administering a dosage amount of the dose of gamma-hydroxybutyrate that would be administered to the subject absent the co-administering, and co-administering comprises administering a dosage amount of the dose of divalproex sodium equivalent to the dosage amount that would be administered to the subject absent co-administration with the dose of gamma-hydroxybutyrate.

13. The method of claim 1, wherein the administering comprises administering a reduced dosage amount of the dose of gamma-hydroxybutyrate relative to a dosage amount of the dose of gamma-hydroxybutyrate that would be administered to the subject absent the co-administering, and the co-administering comprises administering a dosage amount of the dose of divalproex sodium equivalent to the dosage amount that would be administered to the subject absent co-administration with gamma-hydroxybutyrate.

14. The method of claim 13, wherein the dose of gamma-hydroxybutyrate is reduced by less than 5% relative to the dosage amount of the dose of gamma-hydroxybutyrate that would be administered to the subject absent the co-administering.

15. The method of claim 13, wherein the dose of gamma-hydroxybutyrate is reduced by at least 20% relative to the dosage amount of the dose of gamma-hydroxybutyrate that would be administered to the subject absent the co-administering.

16. The method of claim 1, wherein the dose of gamma-hydroxybutyrate comprises an immediate-release portion and a modified-release portion, each of which comprises at least some of the gamma-hydroxybutyrate.

17. A method of decreasing excessive daytime sleepiness in a human subject, the method comprising:
orally administering a single nighttime daily dose to the human subject,
wherein the single nighttime daily dose comprises a first salt of gamma-hydroxybutyric acid and a second salt of gamma-hydroxybutyric acid,
wherein the first salt is selected from the group consisting of a sodium salt of gamma-hydroxybutyric acid, a calcium salt of gamma-hydroxybutyric acid, a potassium salt of gamma-hydroxybutyric acid, and a magnesium salt of gamma-hydroxybutyric acid,
wherein the second salt differs from the first salt and is selected from the group consisting of a sodium salt of gamma-hydroxybutyric acid, a calcium salt of gamma-hydroxybutyric acid, a potassium salt of gamma-hydroxybutyric acid, and a magnesium salt of gamma-hydroxybutyric acid, and
wherein the orally administering occurs only once nightly; and
co-administering divalproex sodium.

18. The method of claim 17, wherein the human subject achieves a decrease in excessive daytime sleepiness when measured by the Epworth Sleepiness Scale (ESS), a decrease in excessive daytime sleepiness when measured by the Maintenance of Wakefulness Test, an improved Clinical Global Impression (CGI) rating of sleepiness, or a decrease in weekly cataplexy attacks.

19. A method of decreasing excessive daytime sleepiness in a human subject, the method comprising:
initiating a starting dosage, wherein the initiating comprises orally administering a single nighttime daily dose to the human subject,
wherein the single nighttime daily dose comprises a sodium salt of gamma-hydroxybutyric acid, a calcium salt of gamma-hydroxybutyric acid, a potassium salt of gamma-hydroxybutyric acid, and a magnesium salt of gamma-hydroxybutyric acid, w
herein the orally administering occurs only once nightly; and
co-administering divalproex sodium.

20. The method of claim 19, wherein the human subject achieves a decrease in excessive daytime sleepiness when measured by the Epworth Sleepiness Scale (ESS), a decrease in excessive daytime sleepiness when measured by the Maintenance of Wakefulness Test, an improved Clinical Global Impression (CGI) rating of sleepiness, or a decrease in weekly cataplexy attacks.
